(19) United States
(12) Patent Application Publication
(10) Pub. No.: US 2025/0223337 A1
(43) Pub. Date: Jul. 10, 2025

(54) METHODS OF PREPARING ALBUMIN PREPARATIONS

(71) Applicant: CSL Behring LLC, King of Prussia, PA (US)

(72) Inventors: Charles Arnold, King of Prussia, PA (US); Jessica Erman, King of Prussia, PA (US); Kenneth Maas, King of Prussia, PA (US)

(73) Assignee: CSL Behring LLC, King of Prussia, PA (US)

(21) Appl. No.: 18/853,807

(22) PCT Filed: Apr. 13, 2023
(86) PCT No.: PCT/US2023/018404

§ 371 Date: Oct. 3, 2024

Related U.S. Application Data
(60) Provisional application No. 63/331,092, filed on Apr. 14, 2022.

(51) Int. Cl. C07K 14/765; C07K 1/14; C07K 1/18

(52) U.S. Cl. CPC C07K 14/765; C07K 1/145; C07K 1/18

(57) ABSTRACT

Described are human serum albumin (hSA) preparations prepared from human plasma and methods of preparing them. The preparations have an amount of C1 esterase (C1E) inhibitor activity and exhibit stability against serine protease activity.

### CROSS-REFERENCE TO RELATED APPLICATIONS

This application claims the benefit of and priority to U.S. Provisional Application No. 63/331,092, filed on Apr. 14, 2022, the entire contents of which are hereby incorporated by reference herein.

### FIELD

Described herein are human serum albumin (hSA) preparations prepared from human plasma and methods of preparing them. The preparations have an amount of C1 esterase (C1E) inhibitor activity and exhibit stability against serine protease activity.

### BACKGROUND

Human serum albumin (hSA) preparations are therapeutically useful and widely used to increase blood volume in cardiovascular surgery, among other uses, such as to treat hypovolemia (low blood volume), hypoalbuminemia (low albumin), burns, acute respiratory distress syndrome (ARDS), nephrosis, acute liver failure, and hemolytic disease of the newborn, or in conjunction with renal dialysis and cardiopulmonary bypass surgery.

hSA preparations for therapeutic use can be prepared from collected (donated) human plasma by fractionation. hSA preparations prepared from human plasma typically include prekallikrein activator (PKA) (also called Factor XIIA) as a contaminant. PKA is the activated form of the Hageman factor (Factor XII), which is a zymogen of a serine protease. PKA forms part of a protease cascade and catalyses the conversion of prekallikrein (Factor XI) to kallikrein (Factor XIA), which is another serine protease. Kallikrein acts on kininogen and catalyzes the formation of kinins such as bradykinin. Bradykinin is a physiologically and pharmacologically active peptide of the kinin group of proteins that is a potent endothelium-dependent vasodilator and mild diuretic, which may cause a lowering of blood pressure. During rapid infusion of hSA preparations, an elevated bradykinin content may result in hypotension and angioedema, which may be medical emergencies.

Thus, there is a need for hSA preparations with a low serine protease activity, including hSA preparations with a low PKA activity, and for methods of preparing such hSA preparations.

### SUMMARY

Provided herein are methods of preparing a human serum albumin preparation, comprising extracting an amount of C1 esterase inhibitor (C1E-inhibitor) from an albumin-containing fraction of human plasma to obtain an albumin preparation comprising an amount of C1E-inhibitor effective to inhibit protease activity in the albumin preparation. In some embodiments, the albumin preparation has an albumin content of at least 5% w/v, and, after pasteurization and storage at a temperature of 18-25° C. for 1 month, the albumin preparation has one or both of (i) a serine protease activity of less than or equal to 0.8 nkat/g total protein and (ii) a prekallikrein-activator (PKA) activity of less than or equal to 35 IU/ml total protein. In some embodiments, the method further comprises, in the same extracting step or separate extracting step(s), extracting from the albumin-containing fraction of human plasma one or more additional serine protease inhibitors selected from alpha1-proteinase-inhibitor, antithrombin III, alpha1-antichymotrypsin, alpha2-antiplasmin, alpha2-macroglobulin, inter-alpha-trypsin inhibitor and beta1-anticollagenase (collagenase).

The method may comprise mixing a first albumin-containing fraction of human plasma with a second albumin-containing fraction of human plasma to obtain the albumin preparation, wherein the second albumin-containing fraction of human plasma is obtained by a process comprising extracting an amount of C1E-inhibitor from an albumin-containing fraction of human plasma, and wherein the first albumin-containing fraction of human plasma is not subjected to said extracting. The method may further comprise, prior to the mixing, extracting from the second albumin-containing fraction of human plasma one or more additional serine protease inhibitors selected from alpha1-proteinase-inhibitor, antithrombin III, alpha1-antichymotrypsin, alpha2-antiplasmin, alpha2-macroglobulin, inter-alpha-trypsin inhibitor and beta1-anticollagenase (collagenase).

In some embodiments, the fraction of human plasma subjected to extracting is be cryo-poor plasma. In some embodiments, the fraction of human plasma subjected to extracting is an ethanol fraction of human plasma of a human plasma fractionation process.

In some embodiments, the fraction of human plasma subjected to extracting is selected from:
Supernatant I of a Cohn/Oncley industrial plasma fractionation or an equivalent fraction of a different plasma fractionation process;
Supernatant II+III of a Cohn/Oncley industrial plasma fractionation or an equivalent fraction of a different plasma fractionation process;
Supernatant I+II+III of a Cohn/Oncley industrial plasma fractionation or an equivalent fraction of a different plasma fractionation process;
Fraction I+II+III+IV filtrate of a Cohn/Oncley industrial plasma fractionation or an equivalent fraction of a different plasma fractionation process;
Supernatant IV of a Cohn/Oncley industrial plasma fractionation or an equivalent fraction of a different plasma fractionation process;
Supernatant IV1 of a Cohn/Oncley industrial plasma fractionation or an equivalent fraction of a different plasma fractionation process;
Supernatant IV4 of a Cohn/Oncley industrial plasma fractionation or an equivalent fraction of a different plasma fractionation process;
Fraction V precipitate of a Cohn/Oncley industrial plasma fractionation or an equivalent fraction of a different plasma fractionation process.

In some embodiments, the fraction of human plasma subjected to extracting is selected from:
Supernatant A of a Kistler/Nitschmann industrial plasma fractionation;
Filtrate A of a Kistler/Nitschmann industrial plasma fractionation;
Fraction I+II+III suspension of a Kistler/Nitschmann industrial plasma fractionation;
Supernatant IV of a Kistler/Nitschmann industrial plasma fractionation; and
Precipitate C of a Kistler/Nitschmann industrial plasma fractionation.

In any embodiments, the extracting may comprise adsorption on a solid support. In some embodiments, the adsorption on a solid support only partially removes CIE-inhibitor present in the albumin-containing fraction subject to extracting. In some embodiments, the adsorption on a solid support only partially removes one or more additional protease inhibitors present in the albumin-containing fraction subject to extracting. In some embodiments, the extracting comprises a chromatography step on a strong anion exchange resin, optionally wherein the strong anion exchange resin comprises quaternary amino ethyl (QAE) groups.

In some embodiments, the method may comprise, prior to the extracting, subjecting the fraction of human plasma subject to extracting to a chromatography step on a weak anion exchange resin to remove Prothrombin Complex (PTC), optionally wherein the weak anion exchange resin comprises diethyl aminoethyl (DEAE) groups.

In some embodiments, the first and second albumin-containing fractions of human plasma subjected to mixing are filtrates of the same fraction(s) of the same plasma fractionation process. In some embodiments, the first and second albumin-containing fractions of human plasma subjected to mixing are Fraction V precipitates. In some embodiments, the fraction of human plasma subjected to extracting is cryo-poor plasma, and the method further comprises fractionating the extracted cryo-poor plasma to obtain Fraction V precipitate, wherein the first and second albumin-containing fractions of human plasma subjected to mixing are Fraction V precipitates. In some embodiments, the fraction of human plasma subjected to extracting is Fraction V precipitate, optionally wherein the Fraction V precipitate is resuspended to obtain a solution or suspension prior to the extracting, and wherein the first and second albumin-containing fractions of human plasma subjected to mixing are Fraction V precipitates. In some embodiments, the fraction of human plasma subjected to extracting is Fraction V suspension. In some embodiments, the fraction of human plasma subjected to extracting is Fraction C precipitate, optionally wherein the Fraction C precipitate is resuspended to obtain a solution or suspension prior to the extracting, and wherein the first and second albumin-containing fractions of human plasma subjected to mixing are Fraction C precipitates.

In some embodiments, the weight:weight ratio of the first and second albumin-containing fractions of human plasma subjected to mixing is from 10:90 to 90:10. In some embodiments, the weight:weight ratio of the first and second albumin-containing fractions of human plasma subjected to mixing is selected from 10:90, 15:85, 20:80, 25:75, 30:70, 33:67, 40:60, 50:50, 60:40, 67:33, 70:30, 75:25, 80:20, 85:15, and 90:10. In some embodiments, the weight:weight ratio of the first and second albumin-containing fractions of human plasma subjected to mixing is selected from 10:90, 15:85, 20:80, 25:75, 30:70, 33:67, and 40:60. In some embodiments, the weight:weight ratio of the first and second albumin-containing fractions of human plasma subjected to mixing is from about 25:75 to about 33:67. In some embodiments, the weight:weight ratio of the first and second albumin-containing fractions of human plasma subjected to mixing is about 25:75. In some embodiments, the weight:weight ratio of the first and second albumin-containing fractions of human plasma subjected to mixing is about 33:67.

In any embodiments, the method may further comprise subjecting the albumin preparation to pasteurization.

In some embodiments, the method comprises mixing a first albumin-containing fraction of human plasma with a second albumin-containing fraction of human plasma at a weight:weight ratio of about 33:67 to obtain the albumin preparation, wherein the first and second albumin-containing fractions of human plasma are Fraction V precipitates of a Cohn/Oncley industrial plasma fractionation or equivalent fractions of a different plasma fractionation process, wherein the second albumin-containing fraction of human plasma is obtained by a process comprising (i) a chromatography step on a weak anion exchange resin to remove Prothrombin Complex (PTC), optionally wherein the weak anion exchange resin comprises DEAE groups; (ii) extracting an amount of C1E-inhibitor by a chromatography step on a strong anion exchange resin, optionally wherein the strong anion exchange resin comprises quaternary amino ethyl (QAE) groups, (iii) optionally, extracting one or more additional serine protease inhibitors selected from alpha1-proteinase-inhibitor, antithrombin III, alpha1-antichymotrypsin, alpha2-antiplasmin, alpha2-macroglobulin, inter-alpha-trypsin inhibitor and beta1-anticollagenase (collagenase), and, (iv) if the albumin-containing fraction used at step (i) is not a Fraction V precipitate of a Cohn/Oncley industrial plasma fractionation or an equivalent fraction of a different plasma fractionation process, fractionating the fraction to obtain a Fraction V precipitate or an equivalent fraction of a different plasma fractionation process; the first albumin-containing fraction of human plasma is not subjected to said extracting; and pasteurizing the albumin preparation.

In some embodiments, the albumin preparation has an albumin content of at least 5% w/v, and, after pasteurization and storage at a temperature of 18-25° C. for 1 month, the albumin preparation has a PKA activity of less than or equal to 20 IU/ml. In some embodiments, the albumin preparation has an albumin content of at least 5% w/v, a C1E-inhibitor activity of greater than 0.0025 IU per kg total protein, and a PKA activity of less than or equal to 35 IU/ml.

In some embodiments, the method comprises mixing a first albumin-containing fraction of human plasma with a second albumin-containing fraction of human plasma to obtain the albumin preparation, wherein the first albumin-containing fraction of human plasma is obtained by a process comprising extracting a first amount of CIE-inhibitor from an albumin-containing fraction of human plasma, and wherein the second albumin-containing fraction of human plasma is obtained by a process comprising extracting a second amount of CIE-inhibitor from an albumin-containing fraction of human plasma. Also provided are human albumin preparations prepared by such a method.

Also provided herein are human albumin preparation having an albumin content of at least 5% w/v, a CIE-inhibitor activity of greater than 0.0025 IU per kg total protein, and a PKA activity of less than or equal to 35 IU/ml.

Also provided herein are human albumin preparations prepared by a method as described above, wherein, after pasteurization and storage at a temperature of 18-25° C. for a period of time selected from 1 month, 3 months, 6 months, and 12 months, the PKA activity is less than or equal to 20 IU/ml, or less than or equal to 10 IU/ml, or less than or equal to 5 IU/ml.

Also provided herein are human albumin preparations prepared by a method as described above, wherein, after pasteurization and storage at a temperature of 18-25° C. for a period of time selected from 1 month, 3 months, and 6 months, the serine protease activity is less than or equal to 8.0 nkat/g total protein, or less than or equal to 0.06 nkat/g total protein, or less than or equal to 0.01 nkat/g total protein.

In some embodiments of any of the forgoing embodiments, the albumin preparation may have an albumin content of about 5% w/v. In some embodiments of any of the forgoing embodiments, the albumin preparation may have an albumin content of about 25% w/v.

### BRIEF DESCRIPTION OF THE DRAWINGS

FIG. 1 is a process flow diagram of different steps that can be used in a method as described herein, using cryo-rich plasma as starting material. The two left arms illustrate steps for the preparation of unadsorbed fractions (from which C1E-inhibitor is not extracted), while the two right arms illustrate steps for the preparation of adsorbed fractions (from which C1E-inhibitor not extracted).

FIG. 2A is a schematic diagram of a Cohn/Oncley industrial plasma fractionation.

FIG. 2B is a schematic diagram of a Kistler/Nitschmann industrial plasma fractionation.

FIG. 3 reports the serine-like protease activity (nkat/g total protein) of Fraction V precipitate hSA preparations as described herein, prepared using different weight:weight ratios of first and second albumin-containing fractions as described herein (0% adsorbed=100:0; 50% adsorbed=50:50, 67% adsorbed=33:67; 75% adsorbed=25:75; 100% adsorbed=0:100), and then prepared at different protein concentrations (13.5%, 5% or 25% albumin). Serine-like protease activity was determined using the chromogenic substrate S-2288™ (Chromogenix) (H-D-Isoleucyl-L-prolyl-L-arginine-p-nitroaniline dihydrochloride) which is sensitive to a broad spectrum of serine proteases.

FIG. 4 reports serine protease activity (nkat/g total protein) in Fraction V precipitates obtained from cryo-poor plasma that had been subjected to adsorption on a diethyl aminoethyl (DEAE) resin, fractionated to obtain Fraction V precipitate, and then prepared at different protein concentrations (13.5%, 5% or 25% albumin).

FIG. 5 reports serine protease activity (nkat/g total protein) in additional Fraction V precipitate hSA preparations prepared as described for FIG. 3 and FIG. 4.

FIG. 6 illustrates the non-linear correlation of C1E-inhibitor activity (IU/kg total protein) to serine protease activity (nkat/g total protein) in unpasteurized 5% albumin hSA preparations discussed in Example 2.

FIG. 7 illustrates the non-linear correlation of C1E-inhibitor activity (IU/kg total protein) to serine protease activity (nkat/g total protein) in unpasteurized 25% albumin hSA preparations discussed in Example 2.

FIG. 8 reports CIE-Inhibitor activity (IU/kg total protein) in Fraction V precipitate hSA preparations as described herein, prepared using different weight:weight ratios of first and second albumin-containing fractions as described herein (0% adsorbed=100:0; 50% adsorbed=50:50, 67% adsorbed=33:67; 75% adsorbed=25:75; 90% adsorbed=10:90; 100% adsorbed=0:100), and then prepared at different protein concentrations (13.5%, 5% or 25% albumin). The second albumin-containing fractions were prepared by subjecting cryo-poor plasma to extraction as described herein (adsorption on diethyl aminoethyl (DEAE) and quaternary aminoethyl (QAE) resins), and then fractionating to obtain Fraction V precipitate.

FIG. 9 reports CIE-Inhibitor activity (IU/kg total protein) in Fraction V precipitate hSA preparations as described herein, prepared using different weight:weight ratios of first and second albumin-containing fractions as described herein (0% adsorbed=100:0; 67% adsorbed=33:67; 75% adsorbed=25:75; 100% adsorbed=0:100), and then prepared at different protein concentrations (13.5%, 5% or 25% albumin). The second albumin-containing fractions were prepared by subjecting cryo-poor plasma to extraction as described herein (adsorption on diethyl aminoethyl (DEAE) and quaternary aminoethyl (QAE) resins), and then fractionating to obtain Fraction V precipitate. The “DEAE adsorbed” samples were prepared as described for FIG. 4.

FIG. 10 reports C1E-Inhibitor activity (IU/kg total protein) in Fraction V precipitate hSA preparations as described herein, prepared using different weight:weight ratios of first and second albumin-containing fractions as described herein (0% adsorbed=100:0; 90% adsorbed=10:90; 99% adsorbed=1:99; 100% adsorbed=0:100), and then prepared at different protein concentrations (13.5%, 5% or 25% albumin). The second albumin-containing fractions were prepared by subjecting cryo-poor plasma to extraction as described herein (adsorption on diethyl aminoethyl (DEAE) and quaternary aminoethyl (QAE) resins), and then fractionating to obtain Fraction V precipitate.

FIG. 11A and FIG. 11B report the serine-like protease activity (FIG. 11A) and PKA activity (FIG. 11B) in unpasteurized 25% albumin preparations prepared from Fraction V precipitate hSA preparations as described herein that were made with different weight:weight ratios of first and second albumin-containing fractions as described herein (0=100:0; 50=50:50; 67=33:67; 75=25:75).

FIG. 12 reports non-activated partial thromboplastin time (NaPTT) in unpasteurized 25% albumin preparations prepared as described for FIGS. 11A-B. FIG. 12 is a box-and-whisker plot of the obtained data, graphically depicting data in quartiles.

### DETAILED DESCRIPTION

Described herein are human serum albumin (hSA) preparations prepared from human plasma and methods of preparing them. The serine protease activity of the preparations may be stable such that, after pasteurization and storage at a temperature of 18-25° C. for 1 month, the preparation has one or both of (i) a serine protease activity of less than or equal to 0.8 nkat/g total protein, including less than or equal to 0.06 nkat/g total protein and less than or equal to 0.01 nkat/g total protein, and (ii) a prekallikrein-activator (PKA) activity of less than or equal to 35 IU/ml, including less than or equal to 20 IU/ml, including less than or equal to 10 IU/ml and less than or equal to 5 IU/ml, optionally wherein the albumin preparation has an albumin content of at least 5% w/v. The hSA preparations may have a CIE-inhibitor activity of greater than 0.0025 IU per kg total protein and a PKA activity of less than or equal to 35 IU/ml, including less than or equal to 20 IU/ml, including less than or equal to 10 IU/ml and less than or equal to 5 IU/ml, optionally wherein the albumin preparation has an albumin content of at least 5% w/v.

The hSA preparations may be made by a method comprising extracting an amount of C1 esterase inhibitor (C1E-inhibitor) from an albumin-containing fraction of human plasma to obtain an albumin preparation comprising an amount of C1E-inhibitor. The methods may comprise mixing a first albumin-containing fraction of human plasma with a second albumin-containing fraction of human plasma, wherein the second albumin-containing fraction of human plasma is obtained by a process comprising extracting an amount of CIE-inhibitor from an albumin-containing fraction of human plasma, and wherein the first albumin-containing fraction of human plasma is not subjected to said extracting. Alternatively, the methods may comprise mixing a first albumin-containing fraction of human plasma with a second albumin-containing fraction of human plasma to obtain the albumin preparation, wherein the first albumin-containing fraction of human plasma is obtained by a process comprising extracting a first amount of CIE-inhibitor from an albumin-containing fraction of human plasma, and wherein the second albumin-containing fraction of human plasma is obtained by a process comprising extracting a second amount of CIE-inhibitor from an albumin-containing fraction of human plasma.

### Definitions

Technical and scientific terms used herein have the meanings commonly understood by one of ordinary skill in the art to which the present disclosure pertains, unless otherwise defined.

As used herein, the singular forms “a,” “an,” and “the” and the like designate both the singular and the plural, unless expressly stated to designate the singular only.

As used herein, the term “about” means that the stated parameter is not limited to the exact number stated. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein, a range described as “X to Y” includes the stated endpoint values, and as disclosing all possible values in between. Thus, as s used herein, ranges are to be construed as shorthand for each and every value falling within the range, including the stated endpoints, and each separate value should be understood to be expressly disclosed herein.

As used in the present disclosure and claims, the term “and/or”, e.g., “X and/or Y” shall be understood to mean X, Y, and X and Y, and shall be understood to provide explicit support for all meanings.

As used in the present disclosure and claims, the word “comprise” and variations such as “comprises” and “comprising”, shall be understood to designate the inclusion of the stated element(s), feature(s), or step(s), without excluding other element(s), feature(s) or step(s).

It also shall be understood that wherever embodiments are described herein with the term “comprising,” analogous embodiments “consisting essentially of” the stated element(s), feature(s), or step(s) also are provided, as are analogous embodiments “consisting of” the stated element(s), feature(s), or step(s).

As used in the present disclosure and claims, the terms “%”, “percent” and “percentage” shall be understood to refer to percent by weight unless the context clearly dictates otherwise.

As used in the present disclosure and claims, the term “about” shall be understood to mean within 10% of the stated value. Thus, for example, “about 10” shall be understood to mean from 9 to 11 inclusive.

Those skilled in the art will appreciate that the present disclosure is susceptible to variations and modifications other than those specifically described. It is to be understood that the disclosure includes all such variations and modifications. Where elements, features, and steps, are described singularly or in combination, the disclosure includes all permutations and combinations of such elements, features, and steps, unless the context clearly dictates otherwise. Any example of the present disclosure shall be understood to apply mutatis mutandis to any other example unless specifically stated otherwise.

### Human Blood Plasma Fractionation

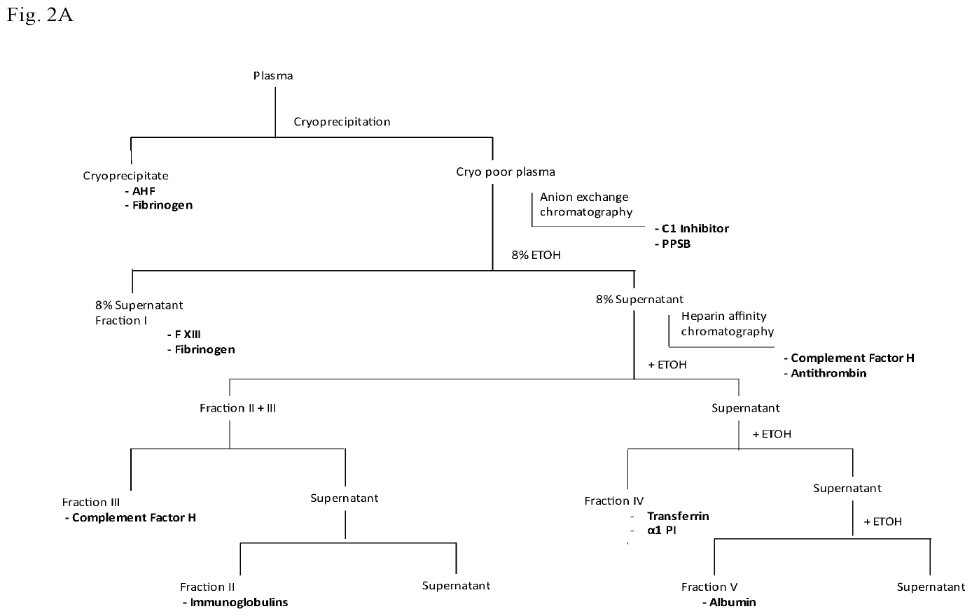

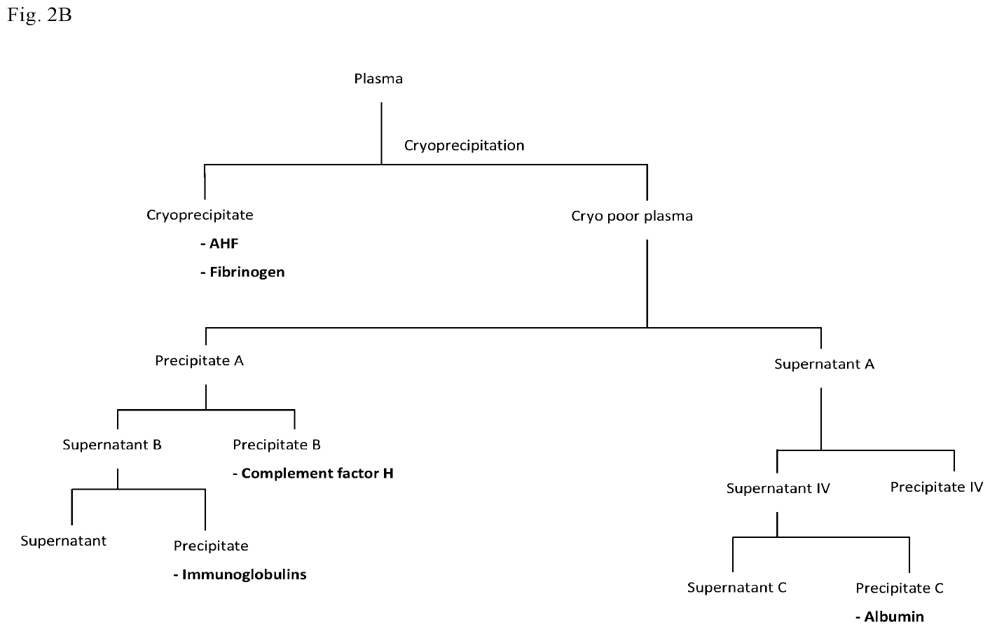

Human blood plasma has been utilized industrially for decades for the manufacture of plasma protein products such as, e.g., human albumin, immunoglobulin preparations (IgG), clotting factor concentrates (such as clotting Factor VIII, clotting Factor IX, prothrombin complex, etc.) and protease inhibitors (such as Antithrombin III, C1 esterase-inhibitor, etc.). Plasma fractionation processes have been developed to obtain these products. In these processes, plasma is sequentially subjected to various physical purification methods (e.g., precipitation, filtration, and adsorption), leading to intermediate products enriched in certain proteins. The separation of individual plasma proteins by fractionation processes can be achieved by exploiting the fact that different plasma proteins have different solubilities depending on, for example, pH, temperature, and ionic strength, as well as different adsorption properties on different types of solid supports (for example). Well-known industrial scale plasma fractionation processes include precipitation with cold ethanol, following protocols such as the Cohn/Oncley fractionation process or Kistler/Nitschmann fractionation process. Typical fractionation methods are reviewed in Schultze and Heremans, MOLECULAR BIOLOGY OF HUMAN PROTEINS. VOLUME I: NATURE AND METABOLISM OF EXTRACELLULAR PROTEINS (Elsevier Publishing Company 1966), p. 236-317. Simplified schematics of such processes are provided in FIG. 2A (Cohn/Oncley) and FIG. 2B (Kistler/Nitschmann).

These industrial scale cold ethanol fractionation processes enable multiple plasma proteins to be extracted from one plasma source. Such processes generally involve as a first step thawing frozen plasma (typically in batch sizes in the range of 1000-15000 kg) to form an albumin-rich supernatant and a precipitate (referred to as a cryoprecipitate) that contains valuable coagulation factors. For example, Factor VIII, Von Willebrand Factor and fibrinogen accumulate in the cryoprecipitate, and can be isolated from the cryoprecipitate by processes including one or more of ethanol precipitation, ion exchange chromatography, affinity chromatography, immunoaffinity, size exclusion chromatography ultrafiltration, and microfiltration. See, e.g., Burnouf, Transfusion Medicine Reviews, vol. 21, pp 101-117 (2007). The albumin-rich supernatant is referred to as cryodepleted plasma, cryosupernatant, or cryo-poor plasma, and itself contains numerous proteins of interest, including albumin. The Cohn/Oncley or Kistler/Nitschmann processes can be used to obtain albumin from the cryosupernatant (cryo-poor plasma).

FIG. 2A provides a flow chart of a Cohn/Oncley fractionation process, which is briefly described. After cryoprecipitation, cryo-poor plasma optionally may be subjected to, e.g., anion exchange chromatography to remove, e.g., C1E-inhibitor and/or human prothrombin complex PPSB. Other steps of FIG. 2A are described in turn below. (i) After cryoprecipitation, the pH of cryo-poor plasma is adjusted to 7.2, ethanol concentration is increased to 8% (by weight of the suspension), and the temperature of the suspension is adjusted to −1° C. The typical precipitate of this step (known as Fraction I) comprises mainly Factor XIII and fibrinogen. The filtrate supernatant (known as Supernatant I filtrate) is separated from the precipitate. (ii) Supernatant I filtrate is subjected to a further fractionation step that involves adjusting the pH to 6.9, increasing the ethanol concentration to 20%, and adjusting the temperature to-5° C. The typical precipitate of this step (known as Fraction II+III) mainly comprises immunoglobulins. (iii) The supernatant of step (ii) is subjected to further fractionation that involves adjusting the pH to 5.2 (maintaining the ethanol content at 20% and the temperature at-5° C.). The typical precipitate of this step (known as Fraction IV-1 or Fraction IV1) mainly contains α1-proteinase inhibitor (α1-antitrypsin) and Antithrombin III. (iv) The supernatant of step (iii) is subject to further fractionation that involves adjusting the pH to about 6 (including to pH 5.8 or pH 5.9) and increasing the ethanol concentration to 40% (maintaining the temperature at −5° C.). The typical precipitate of this step (known as Fraction IV) comprises protein C and α2-macroglobulin. (v) The supernatant of step (iv) is subject to further fractionation that involves adjusting the pH to 4.8, maintaining the ethanol concentration at 40%, and optionally reducing the temperature to −6° C. The typical precipitate of this step (known as Fraction V) contains human serum albumin. In a final step, albumin is precipitated near its isoelectric point. The precipitate paste (Fraction V of Cohn/Oncley or Precipitate C of Kistler/Nitschmann) can be held frozen before further processing. Although most albumin is obtained in Fraction V/Precipitate C, albumin may be present in other fractions. Thus, it also is possible to isolate albumin from other fractions.

While this general summary is provided to provide context for the methods and products described herein, those skilled in the field will understand and appreciate that these processes have some adaptability and have been optimized and varied over the years, for example, to suit different manufacturers and different product profile goals. An example of such a modification is the presence or absence of Cohn fractionation step IV-1, which can be used to extract alpha-1-antitrypsin. Thus, it should be understood that the methods and products described herein can be practiced with modifications and variations of human plasma fractionation processes, and that such modifications and variations are included within the scope of this disclosure.

### Prekallikrein Activator (PKA) and hSA Preparation Stability

Once obtained, hSA preparations often must be stored over extended periods of time prior to use to prepare commercial products, and commercial products may be stored, transported, and stored again prior to administration to patients. Therefore, the stability of hSA preparations over extended periods of time is important to a safe and effective product.

As noted above, however, hSA preparations typically contain prekallikrein activator (PKA) as a contaminant, which can lead to the formation of, for example, bradykinin, via a protease cascade. While not wanting to be bound by theory, it is believed that the PKA present in hSA preparations is generated from Factor XII, such as by surface contact with material surfaces during the fractionation process. Unfortunately, the level of PKA in hSA preparations may increase over storage. While the problems associated with PKA can be addressed to some extent by reducing the PKA activity of an hSA preparation, such as by reducing the content of one or both of Factors XII and XIIA (PKA), the present inventors surprisingly found that the removal of protease inhibitors naturally present in plasma (such as C1E-inhibitor) is associated with an undesired increase in protease activity (such as PKA activity, kallikrein-like protease activity, and other types of serine protease activity), which may be observed during the fractionation process and/or in the final product. While not wanting to be bound by theory, it is believed the loss of protease inhibitors due to the removal of protease inhibitors such as CLE-inhibitor by, for example, resin adsorption steps, permits activation of the protease cascade during the fractionation process and/or during storage periods. Even though pasteurization may eliminate most protease activity, a threshold amount may remain that can lead to activation of a protease/coagulation cascade over time.

The methods described herein address this problem, and provide methods that obtain hSA preparations with stable, low serine protease activity by controlling the levels of serine protease inhibitors in the hSA preparations. Without being bound by any one theory or mode of action, it is believed that the presence of CIE-inhibitor in an hSA preparation as described herein may quench protease activity which otherwise would be present in the final product after pasteurization, and thereby prevent activation of a protease/coagulation cascade.

### Methods of Preparing Stable hSA Preparations

As noted above, described herein are methods of preparing human albumin preparations from human plasma (hSA preparations). The methods can be practiced in conjunction with known fractionation processes, i.e., by modifying known fractionation processes along the lines described herein. For example, the methods described herein can be practiced in conjunction with an ethanol fractionation according to the Cohn/Oncley process (illustrated in FIG. 2A), or a variation thereof, such as a Kistler-Cohn process or Kistler-Nitschmann process (illustrated in FIG. 2B).

Plasma for plasma fractionation can be obtained by plasmapheresis. Donated plasma can be frozen at −25° C. or lower, such as −80° C., and stored prior to use.

Typically, the first step in a fractionation process will be cryoprecipitation. Typically, donated plasma from several donors is pooled in a temperature-controlled container at about +1° C. Under those conditions, not all of the proteins present in plasma dissolve immediately. Thus, a cryoprecipitate forms, and can be separated from the rest of the plasma by centrifugation, for example. As noted above, most of the albumin remains in the supernatant (referred to as cryodepleted plasma or cryo-poor plasma), which can be subjected fractionation to obtain an hSA preparation, such as the ethanol fractionation steps of the Cohn/Oncley process or variants thereof, such as the Kistler/Nitschmann process. As noted above, those processes typically involve removing other proteins, optionally including protease inhibitors such as CIE-inhibitor, in order to obtain an hSA preparation.

While some known methods may include a step that removes all or substantially all C1E-inhibitor (such as an adsorption step on a strong anion exchange resin), the methods described herein comprise extracting an amount of CIE-inhibitor from an albumin-containing fraction of human plasma to obtain an albumin preparation comprising an amount of C1E-inhibitor, such as an amount effective to inhibit protease activity in the hSA preparation. Thus, in some embodiments, a method as described herein comprises retaining an amount of CIE-inhibitor and, optionally, one or more other serine protease inhibitors present in cryo-poor plasma, in order to control (limit) serine protease activity in the hSA preparation. The methods may comprise removing only a portion of C1E-inhibitor from an albumin-containing fraction of human plasma. Additionally or alternatively, the methods may comprise mixing albumin-containing fractions of human plasma that have different amounts of C1E-inhibitor activity. For example, the methods may comprise mixing a first albumin-containing fraction of human plasma with a second albumin-containing fraction of human plasma, wherein the second albumin-containing fraction of human plasma is obtained by a process comprising extracting C1E-inhibitor from an albumin-containing fraction of human plasma, and wherein the first albumin-containing fraction of human plasma is not subjected to said extracting. Alternatively, the methods may comprise mixing a first albumin-containing fraction of human plasma with a second albumin-containing fraction of human plasma to obtain the albumin preparation, wherein the first albumin-containing fraction of human plasma is obtained by a process comprising extracting a first amount of C1E-inhibitor from an albumin-containing fraction of human plasma, and wherein the second albumin-containing fraction of human plasma is obtained by a process comprising extracting a second amount of C1E-inhibitor from an albumin-containing fraction of human plasma, wherein the first and second amounts may be the same or different (e.g., each being about 50% of the C1E-inhibitor present in the respective fraction; the first being about 25% and the second being about 75%; the first being about 10% and the second being about 90%; the first being about 1% and the second being about 99%; the first being any amount less than the second amount, the second amount being any amount up to 100%, etc.).

In some embodiments, the method further comprises extracting an amount of one or more other serine protease inhibitors, for example, one or more selected from alpha1-proteinase-inhibitor, antithrombin III, alpha1-antichymotrypsin, alpha2-antiplasmin, alpha2-macroglobulin, inter-alpha-trypsin inhibitor, beta1-anticollagenase (collagenase). The extraction of an amount of one or more other serine protease inhibitors may be effected in the same step as the extraction of C1E-inhibitor, or in a separate step. For example, alpha1-proteinase-inhibitor may be extracted in a separate step from C1E-inhibitor, as may antithrombin III, as may others.

As noted above, while not wanting to be bound by theory, the methods described herein allow the protease activity in hSA preparations to be reduced by controlling the level of serine protease inhibitor activity present at various stages of the fractionation process, and in hSA preparations obtained by the methods. While some known fractionation processes may remove all or substantially all serine protease inhibitors, the present inventors surprisingly found that doing so can be disadvantageous and that providing some level of serine protease inhibitor activity can result in a more stable product. Thus, while some known fractionation processes may remove all or substantially all serine protease inhibitors at an early step in the fractionation process, such as from cryo-poor plasma, the present inventors surprisingly found that providing or retaining some level of serine protease inhibitor activity can result in a more stable product.

In embodiments that comprise mixing a first albumin-containing fraction of human plasma with a second albumin-containing fraction of human plasma (referred to herein as “mixing” embodiments), the second albumin-containing fraction of human plasma is obtained by a process comprising extracting an amount of C1E-inhibitor from an albumin-containing fraction of human plasma, while, in most embodiments, the first albumin-containing fraction of human plasma is not subjected to said extracting. In such embodiments, all or substantially all of the C1E-inhibitor present may be removed from the second albumin-containing fraction, because C1E-inhibitor is not removed from the first albumin-containing fraction. When the second albumin-containing fraction is mixed with the first albumin-containing fraction that had not been subjected to said extracting, the resulting mixture comprises an amount of CIE-inhibitor. As noted above, in alternative embodiments, a first amount of C1E-inhibitor is extracted from the first fraction and a second amount of CIE-inhibitor is extracted from the second fraction. In accordance with any embodiments, the mixing ratio can be selected to achieve one or more of a desired level of CIE-inhibitor concentration in the mixed composition, a desired level of serine protease inhibitor activity in the mixed composition, or to maintain serine protease inhibitor activity in the mixed composition at or below a desired level, as discussed in more detail below.

The following discussion is focused on embodiments where the first albumin-containing fraction of human plasma is not subjected to said extracting. It will be understood that similar principals, methods, and features will apply to the alternative embodiments wherein a first amount of CIE-inhibitor is extracted from the first fraction and a second amount of CIE-inhibitor is extracted from the second fraction.

The “second albumin-containing fraction” may be referred to herein as “adsorbed material,” to signify that the fraction has been subjected to one or more adsorption steps to remove an amount of CIE-inhibitor. The second albumin-containing fraction comprises a lower amount, including a minor amount, of CIE-inhibitor, or is depleted of CIE-inhibitor. In specific embodiments, all or substantially all C1E-inhibitor is removed from the second albumin-containing fraction.

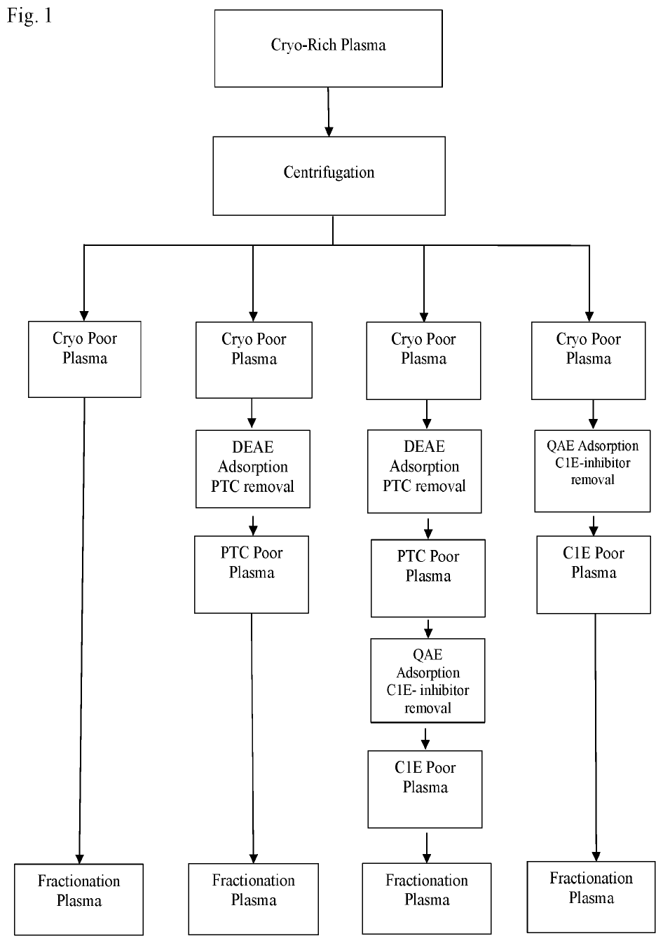

The “first albumin-containing fraction” may be referred to herein as “unadsorbed material,” to signify that the fraction has not been subjected to one or more adsorption steps to remove an amount of C1E-inhibitor. In some embodiments, the “first albumin-containing fraction” is or has been subjected to one or more adsorption steps to remove PTC complex, such as adsorption on a weak ion exchange resin, such as a DEAE resin as discussed in more detail below and illustrated in FIG. 1.

As discussed above, by mixing the first albumin-containing fraction with the second albumin-containing fraction, the serine protease inhibitor activity of the hSA preparation can be regulated.

Extracting an amount of C1E-inhibitor, and, optionally, one or more other serine protease inhibitors, can be achieved by using various methods by which the specific inhibitor of interest can be removed (totally or in partially) from the albumin-containing fraction of human plasma. The terms “extracting” and “extraction” as used herein refer to any separation process. For example, suitable methods include one or more of precipitation, filtration, extraction, and adsorption on a solid support, such as chromatography, such as ion exchange chromatography or affinity chromatography on a solid support, such as by adsorption on a solid support. In some embodiments, the extraction comprises a chromatography step, such as may be carried out by adsorption on a solid support, such as a resin or matrix. For example, an amount of CIE-inhibitor, and, optionally, one or more other serine protease inhibitors (in the same step or separate step(s)), can be extracted by ion exchange chromatography or affinity chromatography on a solid support, as discussed in more detail below.

Chromatographic extraction processes typically employ a solid support, also referred to interchangeably herein as a resin or matrix. Examples of suitable solid supports include inorganic carriers, such as glass and silica gels, organic, synthetic, and naturally occurring carriers, such as agarose, cellulose, dextran, polyamide, polyacrylamides, vinyl copolymers of bifunctional acrylates, various hydroxylated monomers, and the like. Commercially available carriers are sold under the names SEPHADEX™, SEPHAROSE™, HYPERCEL™, CAPTO™, FRACTOGEL™ MACROPREP™, UNOSPHERE™, GIGACAP™, TRISACRYL™, ULTROGEL™, DYNOSPHERES™, MACROSORB™ AND XAD™. Alternatively, an anion exchange chromatography membrane can be used instead of an anion exchange chromatography resin. Suitable commercially available anion exchange membranes include SARTOBIND™ Q (Sartorius), MUSTANG™ Q (Pall Technologies) and INTERCEPT™ Q (Millipore). Several exchange media can be used, such as one or more of a size-exclusion resin and an ion exchange resin, such as an anion exchange resin.

In certain embodiments, the extracting may comprise adsorption on a solid support, which refers to the use of anion exchange resin (or membrane), also called anion exchanger, wherein the target protein is adsorbed to the resin (or membrane). One or both of a strong anion exchange resin and a weak anion exchange resin may be used. The terms “strong” and “weak” refer to the acidic/basic properties of the functional group on the resin. If the ligand is derived from a strong acid or a strong base it is referred to as a “strong” ion exchange resin. If the ligand is derived from a weak acid or a weak base it is referred to as a “weak” ion exchange resin. For example, if a resin is functionalized with a quaternary amine, which is considered a strong base, the resin is called a strong anion exchange resin. Strongly basic anion resins maintain their negative charge across a wide pH range, whereas weakly basic anion resins are neutralized at higher pH levels. Thus, depending on the target protease inhibitor(s) being removed and target extent of removal (total or partial), the anion exchange resin may be a strong anion exchanger (i.e., strongly basic) or a weak anion exchanger (i.e., weakly basic). Typically, the anion exchange resin comprises basic moieties, such as quaternary ammonium or ammine groups. In certain embodiments, a method as described herein comprises at least one chromatography step using an anion exchange resin. In certain embodiments, the method comprises two chromatography steps using an anion exchange resin. In certain embodiments, the method comprises three chromatography steps using an anion exchange resin. In certain embodiments, a method as described herein comprises a chromatography step on a strong anion exchange resin. In certain embodiments, the method comprises only one chromatography step, typically on a strong anion exchange resin.

In any embodiments using a strong anion exchange resin, the strong anion exchange resin may comprise a quaternary amine functional ligand (e.g., —N+(CH₃)₃, such as the MACRO-PREP™ HIGH Q resin (Bio-Rad Laboratories). Additionally or alternatively, in any embodiments using a strong anion exchange resin, the strong anion exchange resin may comprise trimethylamine groups, which may be grafted to a hydroxylated methacrylic polymer via a linking group such, as TOYOPEARL® GIGACAP Q-650M (Tosoh Bioscience LLC). Additionally or alternatively, in any embodiments using a strong anion exchange resin, the strong anion exchange resin may comprise quaternary amino ethyl (QAE) groups, referred to herein as a QAE resin. An example of a suitable QAE resin is a QAE SEPHADEX® resin (Sigma Aldrich) (including the A-50 and A-25 products), which may have a bead size of 40 to 125 μm in a dry state. Adsorption on a QAE resin is typically performed at 10-25° C., preferably at 15-20° C., more preferably at 15° C. Other, non-limiting examples of suitable strong anion exchange resins include POROS™ HQ 10, POROS™ HQ 20, POROS™ HQ 50, and POROS™ XQ from ThermoFisher; Mono Q®, Source™ 15Q, Source™ 30Q, Q SEPHAROSER High Performance, QAE SEPHADEX® and Q SEPHAROSER Fast Flow from Cytiva; WP QUAT™ from J. T. Baker, Hydrocell™ QA from Biochrom Labs Inc.; UNOsphere™ Q and Macro-Prep™ High Q from Biorad; Q-Ceramic HYPERD®, Q HYPERZ®, QMA SPHEROSIL® LS, and QMA SPHEROSIL® M from Pall Technologies; DOWEX® Fine Mesh Strong Base Type I and Type II Anion Matrix from Dow Liquid Separations; CELLUFINE® Q500 and CELLUFINE® Q800 from Millipore; Amberlite™ strong anion exchangers type I and II, DOWEX™ strong anion exchangers type I and II, Diaion™ strong anion exchangers type I and II, TSKgel® Q, TOYOPEARL® SuperQ-650S, TOYOPEARL® SuperQ-650M and TOYOPEARL® SuperQ-650C3, and TOYOPEARL® QAE-26-550C from Tosoh, and QA52™ and Express-Ion™ Q from Whatman. As noted above, an anion exchange chromatography membrane can be used. Suitable commercially available anion exchange membranes include, but are not limited to, SARTOBIND® Q from Sartorius, MUSTANG® Q from Pall Technologies and INTERCEPT® Q membrane from Millipore.

A strong anion exchanger can be used to extract more than 90%, more than 95%, or more than 99% of one or more serine protease inhibitors, such as CIE-inhibitor, and can therefore remove more than 90%, more than 95%, or more than 99% of the respective inhibitor activity, including all or substantially all of the CIE-inhibitor activity. In certain embodiments, the anion exchanger completely extracts one or more serine protease inhibitors, such as CIE-inhibitor, and removes about 100% of the serine protease inhibitor activity.

In certain embodiments, a method as described herein may comprise a chromatography step on a weak anion exchange resin. Non-limiting examples of suitable commercially available weak anion exchange resins include resins with diethylamino ethyl (DEAE) or dimethylethanolamine (DMAE) groups, such as DEAE®, POROS® 50 D, POROS® 50 PI or DOWEX®. In certain embodiments using a weak anion exchange resin, the weak anion exchange resin is a DEAE resin, such as a DEAE-SEPHAROSE® resin, which has a bead size of 40 to 125 μm in a dry state, a pore size of about 200,000 Da exclusion limit, and a capacity of 3-4 meq/g binding capacity. Adsorption on a DEAE resin is typically performed at 15-25° C., preferably at 20° C. As noted above, in mixing embodiments, the “unadsorbed” fraction can be adsorbed on a DEAE resin to remove PTC complex.

Thus, a method as described herein may comprise one or more chromatography steps using an anion exchange resin. For example, a method as described herein may comprise at least one chromatography step using a strong anion exchange resin to extract C1E-inhibitor. When more than one chromatography step is used, one step may use a strong anion exchange resin, and the other(s) may independently use a strong anion exchange resin or a weak anion exchange resin. Those skilled in the art can select and use suitable resins based on the target protein(s) being removed and the desired extent of removal (e.g. total or partial).

FIG. 1 illustrates different steps that can be used in a method as described herein, using cryo-rich plasma as starting material. The two left arms illustrate steps for the preparation of unadsorbed fractions (from which CIE-inhibitor is not extracted), while the two right arms illustrate steps for the preparation of adsorbed fractions (from which CIE-inhibitor not extracted). The left most arm illustrates the preparation of cryo-poor plasma followed by fractionation (e.g., one or more fractionation steps). The next arm to the right illustrates the preparation of cryo-poor plasma, followed by adsorption to DEAE resin to extract PTC complex (and obtain PTC plasma), followed by fractionation (e.g., one or more fractionation steps). The next arm to the right illustrates the preparation of cryo-poor plasma followed by adsorption to DEAE resin to extract PTC complex (and obtain PTC-poor plasma), followed by adsorption to QAE resin to extract C1E-inhibitor (and obtain CIE-inhibitor-poor plasma), followed by fractionation (e.g., one or more fractionation steps). The right-most arm illustrates the preparation of cryo-poor plasma followed by adsorption to QAE resin to extract C1E-inhibitor (and obtain CIE-inhibitor-poor plasma), followed by fractionation (e.g., one or more fractionation steps).

In certain embodiments, the method described herein comprise an adsorption step to remove one or more serine protease inhibitors, such as C1E-inhibitor, using a strong anion exchange resin (such as a QAE resin). The substrate for such an adsorption step may be cryo-poor plasma or Fraction V precipitate, or any other albumin-containing fraction, as discussed in more detail below. Use of a strong anion exchange resin may completely remove CIE-inhibitor (and, in some embodiments, other serine protease inhibitors) from the albumin-containing fraction.

In certain embodiments, the methods additionally comprise, typically prior to adsorption on a strong anion exchange resin, adsorption on a weak ion exchange resin (such as a DEAE resin), to remove factors of the prothrombin complex (PTC), such as one or more or all of Factor II, Factor VII, Factor IX, and Factor X.

In certain embodiments, the extraction of one or more serine protease inhibitors (i.e., C1E-inhibitor and, optionally, other serine protease inhibitors in the same step or in separate step(s)) removes all or substantially all of the respective inhibitor activity present in the fraction subjected to extraction, such as all or substantially all C1E-inhibitor, and, optionally, all or substantially all other serine protease inhibitors present in the fraction subjected to extraction.

In certain embodiments, the extraction of one or more serine protease inhibitors (i.e., C1E-inhibitor and, optionally, other serine protease inhibitors in the same step or in separate step(s)) removes 90% or less of the respective inhibitor activity present in the fraction subjected to extraction. In certain embodiments, the extraction removes 85%, 80%, 75%, 70%, 65%, 60%, 55%, 50%, 45%, 40%, 35%, 30%, 25%, 20%, or 15%, or less of the respective inhibitor activity. In specific embodiments, the extraction removes about 75% or less of the respective inhibitor activity, including 75%. In further specific embodiments, the extraction removes about 67% or less of the respective inhibitor activity, including 67%.

In certain embodiments, the extraction of CIE-inhibitor removes 90% or less of C1E-inhibitor activity present in the fraction subjected to extraction. In certain embodiments, the extraction of CIE-inhibitor removes 85%, 80%, 75%, 70%, 65%, 60%, 55%, 50%, 45%, 40%, 35%, 30%, 25%, 20%, or 15%, or less of C1E-inhibitor activity. In specific embodiments, the extraction of C1E-inhibitor removes 90% or less of C1E-inhibitor activity, including 90%. In specific embodiments, the extraction of C1E-inhibitor removes about 75% or less of C1E-inhibitor activity, including 75%. In further specific embodiments, the partial extraction of C1E-inhibitor removes about 67% or less of CIE-inhibitor activity, including 67%.

In certain embodiments, the extraction of one or more serine protease inhibitors, removes 10% to 90% of the respective inhibitor activity, such as 10% to 90% of C1E-inhibitor activity. In certain embodiments, the extraction of one or more serine protease inhibitors removes 75% to 90% of the respective inhibitor activity, such as 75% to 90% of C1E-inhibitor activity. In certain embodiments, the extraction of one or more serine protease inhibitors removes 15% to 75% of the respective inhibitor activity, such as 15% to 75% of C1E-inhibitor. In certain embodiments, the extraction of one or more serine protease inhibitors removes 30% to 75% or 25% to 75% of the respective inhibitor activity, such as 30% to 75% or 25% to 75 of C1E-inhibitor activity. In certain embodiments, the extraction of one or more serine protease inhibitors removes 33% to 67% of the respective inhibitor activity, such as 33% to 67% of C1E-inhibitor activity. In certain embodiments, the extraction of one or more serine protease inhibitors removes 10% to 70% of the respective inhibitor activity, such as 10% to 70% of C1E-inhibitor activity. In certain embodiments, the extraction of C1E-inhibitor removes 15% to 70% of C1E-inhibitor activity. In certain embodiments, the extraction of CIE-inhibitor removes 25% to 70% of C1E-inhibitor activity. In certain embodiments, the extraction of CIE-inhibitor removes 30% to 70% of C1E-inhibitor activity. In specific embodiments, the extraction of CIE-inhibitor removes 33% to 67% of C1E-inhibitor activity. In further specific embodiments, the extraction of CIE-inhibitor removes 75% to 90% of CIE-inhibitor activity.

As noted above, the albumin-containing fraction that is subjected to extracting to remove, e.g., C1E-inhibitor, can be any fraction of a human serum fractionation process, from the initially obtained cryo-poor plasma to the final Fraction V precipitate of a Cohn/Oncley industrial plasma fractionation, or equivalent fractions of a different plasma fractionation process. Thus, the extracting can be carried out with a fraction obtained at any stage of a fractionation process.

In some embodiments, the fraction of human plasma subjected to extracting is or comprises cryo-poor plasma.

In some embodiments, the fraction of human plasma subjected to extracting is or comprises an ethanol fraction of human plasma.

In some embodiments, the fraction of human plasma subjected to extracting is or comprises a fraction selected from:
Supernatant I of a Cohn/Oncley industrial plasma fractionation or an equivalent fraction of a different plasma fractionation process;
Supernatant II+III of a Cohn/Oncley industrial plasma fractionation or an equivalent fraction of a different plasma fractionation process;
Supernatant I+II+III of a Cohn/Oncley industrial plasma fractionation or an equivalent fraction of a different plasma fractionation process;
Fraction I+II+III+IV filtrate of a Cohn/Oncley industrial plasma fractionation or an equivalent fraction of a different plasma fractionation process;
Supernatant IV of a Cohn/Oncley industrial plasma fractionation or an equivalent fraction of a different plasma fractionation process;
Supernatant IV1 of a Cohn/Oncley industrial plasma fractionation or an equivalent fraction of a different plasma fractionation process;
Supernatant IV4 of a Cohn/Oncley industrial plasma fractionation or an equivalent fraction of a different plasma fractionation process; and
Fraction V precipitate of a Cohn/Oncley industrial plasma fractionation or an equivalent fraction of a different plasma fractionation process.

In some embodiments, the fraction of human plasma subjected to extracting is or comprises a fraction selected from:
Supernatant A of a Kistler/Nitschmann industrial plasma fractionation;
Filtrate A of a Kistler/Nitschmann industrial plasma fractionation;
Fraction I+II+III suspension of a Kistler/Nitschmann industrial plasma fractionation;
Supernatant IV of a Kistler/Nitschmann industrial plasma fractionation; and
Precipitate C of a Kistler/Nitschmann industrial plasma fractionation.

When the fraction subjected to the extracting comprises Fraction V precipitate or Precipitate C, the fraction optionally may be resuspended to obtain a solution or suspension prior to the extracting.

In some embodiments of mixing embodiments, the second albumin-containing fraction subjected to the extracting is not subjected to further fractionation before mixing with the first albumin-containing fraction. For example, the second albumin-containing fraction may comprise cryo-poor plasma that is extracted to remove C1E-Inhibitor (and, optionally, other serine protease inhibitors), and then mixed a first albumin-containing fraction which also comprises cryo-poor plasma, and then the mixture is subject to further fractionation.

In other embodiments of mixing embodiments, the second albumin-containing fraction subjected to the extracting is a fraction obtained at an early step in the fractionation process, and is subjected to further fractionation before mixing with the first albumin-containing fraction. For example, the second albumin-containing fraction may comprise cryo-poor plasma that is subjected to further fractionation after removal of C1E-Inhibitor (and, optionally, other serine protease inhibitors) such as fractionation to obtain Fraction V precipitate, prior to mixing with a first albumin-containing fraction which also comprises Fraction V precipitate (for example).

In any embodiments of mixing embodiments, the fractions that are mixed typically comprise the same fraction(s) of the same plasma fractionation process.

In some embodiments, the first fraction includes 100% of serine protease inhibitor activity, relative to the serine protease inhibitor activity of the starting material (e.g., donated plasma). In some embodiments, the first fraction includes 100% of C1E-inhibitor activity, relative to the C1E-inhibitor activity of the starting material (e.g., donated plasma). In some embodiments, the first fraction includes less than 100% of serine protease inhibitor activity, relative to the serine protease inhibitor activity of the starting material (e.g., donated plasma). In some embodiments, the first fraction includes less than 100% of C1E-inhibitor activity, relative to the C1E-inhibitor activity of the starting material (e.g., donated plasma). In the latter embodiments, serine protease inhibitor activity (including CIE-inhibitor activity) may be lost during fractionation steps or other processing steps that the first fraction is subjected to prior to the mixing.

As noted above, the mixing ratio can be selected to achieve one or more of a desired level of C1E-inhibitor activity, a desired level of serine protease inhibitor activity, or to maintain serine protease inhibitor activity at or below a desired level over storage. For example, the weight:weight mixing ratio of the first fraction to the second fraction may be from 10:90 to 90:10, such as 10:90, 15:85, 20:80, 25:75, 30:70, 33:67, 40:60, 50:50, 60:40, 67:33, 70:30, 75:25, 80:20, 85:15, or 90:10. In some embodiments, the weight:weight mixing ratio of the first fraction to the second fraction is selected from 10:90, 15:85, 20:80, 25:75, 30:70, 33:67, and 40:60. In some embodiments, the weight:weight mixing ratio of the first fraction to the second fraction is from about 25:75 to about 33:67. In some embodiments, the weight:weight mixing ratio of the first fraction to the second fraction is about 25:75. In some embodiments, the weight:weight mixing ratio of the first fraction to the second fraction is 25:75. In some embodiments, the weight:weight mixing ratio of the first fraction to the second fraction is about 33:67. In certain embodiments, the ratio of the first fraction to the second fraction in the mixture is 33:67.

In specific embodiments, the first and second fractions that are mixed comprise Fraction V precipitate of a Cohn/Oncley industrial plasma fractionation or equivalent fractions of a different plasma fractionation process.

In further specific embodiments, the first and second fractions that are mixed comprise filtrate of ethanol Fractions I, II and III of a Cohn/Oncley industrial plasma fractionation or equivalent fractions of a different plasma fractionation process.

In specific embodiments, a method as described herein comprises mixing cryo-poor plasma that has been adsorbed on a QAE resin to remove CIE-inhibitor at a weight:weight ratio of about 75:25 with cryo-poor plasma that has not been subjected to C1E-inhibitor extraction, typically wherein the cryo-poor plasma that has not been subjected to C1E-inhibitor extraction has not been subjected to any protease inhibitor extraction step.

In other specific embodiments, a method as described herein comprises mixing cryo-poor plasma that has been adsorbed on a QAE resin to remove C1E-inhibitor at a weight:weight ratio of about 67:33 with cryo-poor plasma that has not been subjected to CIE-inhibitor extraction, typically wherein the cryo-poor plasma that has not been subjected to C1E-inhibitor extraction has not been subjected to any protease inhibitor extraction step.

In other embodiments, a method as described herein comprises mixing a first fraction of Fraction V precipitate with a second fraction of Fraction V precipitate at a weight:weight ratio of about 25:75, wherein the second fraction of Fraction V precipitate is obtained by a process comprising adsorbing cryo-poor plasma on a QAE resin to remove CIE-inhibitor and subjecting the extracted cryo-poor plasma to fractionation to obtain Fraction V precipitate, and the first fraction of Fraction V precipitate is obtained by a process that does not comprise extraction of CIE-inhibitor. Typically, the first fraction of Fraction V precipitate is obtained by a process that does not comprise any protease inhibitor extraction step.

In other embodiments, a method as described herein comprises mixing a first fraction of Fraction V precipitate with a second fraction of Fraction V precipitate at a weight:weight ratio of about 33:67, wherein the second fraction of Fraction V precipitate is obtained by a process comprising adsorbing cryo-poor plasma on a QAE resin to remove CIE-inhibitor and subjecting the extracted cryo-poor plasma to fractionation to obtain Fraction V precipitate, and the first fraction of Fraction V precipitate is obtained by a process that does not comprise extraction of C1E-inhibitor. Typically, the first fraction of Fraction V precipitate is obtained by a process that does not comprise any protease inhibitor extraction step.

In other specific embodiments, a method as described herein comprises mixing Fraction V precipitate that has been adsorbed on a QAE resin to remove C1E-inhibitor at a weight:weight ratio of about 75:25 with Fraction V precipitate that has not been subjected to CIE-inhibitor extraction, typically wherein the Fraction V precipitate that has not been subjected to CIE-inhibitor extraction has not been subjected to any protease inhibitor extraction step.

In other specific embodiments, a method as described herein comprises mixing Fraction V precipitate that has been adsorbed on a QAE resin to remove CIE-inhibitor at a weight:weight ratio of about 67:33 with Fraction V precipitate that has not been subjected to C1E-inhibitor extraction, typically wherein the Fraction V precipitate that has not been subjected to CIE-inhibitor extraction has not been subjected to any protease inhibitor extraction step.

In any embodiments, the method may further comprise subjecting the hSA preparation to pasteurization. For example, the method may further comprise subjecting Fraction V hSA preparation to pasteurization. The term “pasteurization” as used herein is a heat treatment. The temperature of the pasteurization step can be about 60° C. The duration of the pasteurization can be 10 hours or longer, including 10 to 11 hours. In certain embodiments, the temperature used in the pasteurization step is about 60° C. and the duration of the heat treatment is 10 to 11 hours. In certain embodiments, for pasteurization the temperature is maintained at about 60° C. for 10 to 13 hours.

### Stable Human Serum Albumin Preparations

As used herein, the terms “human serum albumin preparation” and “hSA preparation” refer to compositions comprising albumin obtained from human blood plasma by a fractionation process, and include solutions of albumin in water or an aqueous solvent, such as a solvent comprising water and ethanol, as well as compositions in which the albumin is only partly dissolved, as well as compositions in which the albumin is present mostly or entirely as a solid. As noted above, an hSA preparation as described herein can optionally be pasteurized.

Typically, an hSA preparation as described herein comprises water, albumin, and amount of C1E-inhibitor, and optionally, one or more other protease inhibitors. The one or more other protease inhibitors may inhibit serine like protease and/or a kallikrein-like proteases, and can be selected from serine protease inhibitors and prekallikrein activator (PKA) inhibitors. More particularly, the one or more other serine protease inhibitors can be selected from alpha1-proteinase-inhibitor, antithrombin III, alpha1-antichymotrypsin, alpha2-antiplasmin, alpha2-macroglobulin, inter-alpha-trypsin inhibitor, and beta1-anticollagenase. As discussed above, an hSA preparation as described herein may be depleted of one or more other protease inhibitors, e.g., may be depleted of one or more or all of alpha1-proteinase-inhibitor, antithrombin III, alpha1-antichymotrypsin, alpha2-antiplasmin, alpha2-macroglobulin, inter-alpha-trypsin inhibitor, and beta1-anticollagenase, either partly, substantially completely, or completely.

Thus, in some embodiments, an hSA preparation as described herein comprises water, albumin, C1E-inhibitor and one or more additional serine protease inhibitors selected from alpha1-proteinase-inhibitor, antithrombin III, alpha1-antichymotrypsin, alpha2-antiplasmin, alpha2-macroglobulin, inter-alpha-trypsin inhibitor, and beta1-anticollagenase.

In some embodiments, an hSA preparation as described herein comprises water, albumin, C1E-inhibitor, and antithrombin III.

As noted above, hSA preparations described herein, e.g., prepared as described herein, comprise an amount of CIE-inhibitor effective to inhibit protease activity in the hSA preparation. In some embodiments, an hSA preparation as described herein comprises CIE-inhibitor activity of greater than 0.0025 IU per kg total protein. Additionally or alternatively, pasteurized hSA preparations as described herein, such as a pasteurized hSA preparation having an albumin content of at least 5% w/v, such as 5% or 25% w/v, may have a PKA activity of less than or equal to 35 IU/ml. In certain embodiments, a pasteurized hSA preparation as described herein, such as a pasteurized hSA preparation having an albumin content of at least 5% w/v, such as 5% or 25% w/v, has a PKA activity of less than or equal to 20 IU/ml. In certain embodiments, a pasteurized hSA preparation as described herein, such as a pasteurized hSA preparation having an albumin content of at least 5% w/v, such as 5% or 25% w/v, has a PKA activity of less than or equal to 10 IU/ml. In certain embodiments, a pasteurized hSA preparation as described herein, such as a pasteurized hSA preparation having an albumin content of at least 5% w/v, such as 5% or 25% w/v, has a PKA activity of less than or equal to 5 IU/ml. For example, a pasteurized hSA preparation as described herein containing 25% w/v albumin may have a PKA activity of less than or equal to 20 IU/ml, equivalent to less than or equal to 80 IU/g total protein. PKA activity can be measured indirectly using an assay as illustrated in the examples, e.g., using chromogenic substrate S-2302™ (Chromogenix) (available from Diapharma) and measuring absorbance at 405 nm (due to enzymatic production of p-nitroaniline).

Additionally or alternatively, the serine protease activity of hSA preparations as described herein may be stable such that, after pasteurization and storage at a temperature of 18-25° C. for 1 month, the preparation has one or both of (i) a serine protease activity of less than or equal to 0.8 nkat/g total protein and (ii) a prekallikrein-activator (PKA) activity of less than or equal to 35 IU/ml. For example, preparations as described herein may exhibit such serine protease activity and/or PKA activity after pasteurization and storage at a temperature of 18-25° C. for 1 month, 3 months, 6 months, 12 months, or longer. Additionally or alternatively, the serine protease activity of preparations as described herein may be stable such that, after pasteurization and storage at a temperature of 18-25° C. for 1 month, the preparation has one or both of (i) a serine protease activity of less than or equal to 0.8 nkat/g total protein and (ii) a prekallikrein-activator (PKA) activity of less than or equal to 20 IU/ml. For example, preparations as described herein may exhibit such serine protease activity and/or PKA activity after pasteurization and storage at a temperature of 18-25° C. for 1 month, 3 months, 6 months, 12 months, or longer.

An hSA preparation as described herein may exhibit a protease inhibitor activity such that the preparation has a serine protease activity of less than or equal to 0.8 nkat/g total protein after pasteurization and storage for 1 month or longer, including 3 months or longer, including 6 months, at a temperature of 18-25° C. In certain embodiments, an hSA preparation as described herein has a serine protease activity of less than or equal to 0.5 nkat/g total protein, after pasteurization and storage for 1 month or longer, including for 3 months or longer, including 6 months, at a temperature of 18-25° C. In certain embodiments, an hSA preparation as described herein has a serine protease activity of less than or equal to 0.1 nkat/g total protein, after pasteurization and storage for 1 month or longer, including for 3 months or longer, including 6 months, at a temperature of 18-25° C. In certain embodiments, an hSA preparation as described herein has a serine protease activity of less than or equal to 0.08 nkat/g total protein, after pasteurization and storage for 1 month or longer, including 3 months or longer, including 6 months, at a temperature of 18-25° C. In certain embodiments, an hSA preparation as described herein has a serine protease activity of less than or equal to 0.06 nkat/g total protein, after pasteurization and storage for 1 month or longer, including 3 months or longer, including 6 months, at a temperature of 18-25° C. In certain embodiments, an hSA preparation as described herein has a serine protease activity of less than or equal to 0.01 nkat/g total protein, after pasteurization and storage for 1 month or longer, including 3 months or longer, including 6 months, at a temperature of 18-25° C. In specific embodiments, an hSA preparation as described herein having an albumin content of about 5% w/v has a serine protease activity of less than or equal to 0.06 nkat/g total protein, after pasteurization and storage for 1 month or longer, including 3 months or longer, including 6 months, at a temperature of 18-25° C. In specific embodiments, an hSA preparation as described herein having an albumin content of about 25% w/v has a serine protease activity of less than or equal to 0.01 nkat/g total protein, after pasteurization and storage for 1 month or longer, including 3 months or longer, including 6 months, at a temperature of 18-25° C.

As noted above, hSA preparations as described herein may have a C1E-inhibitor activity of greater than 0.0025 IU per kg total protein, including after pasteurization. In certain embodiments, a pasteurized or unpasteurized hSA preparation as described herein has a serine protease-inhibitor activity of at least 0.005 IU per kg total protein. In certain embodiments, a pasteurized or unpasteurized hSA preparation as described herein has a C1E-inhibitor activity of at least 0.005 IU per kg total protein. In certain embodiments, a pasteurized or unpasteurized hSA preparation as described herein has a serine protease inhibitor activity of at least 0.0075 IU per kg total protein. In certain embodiments, a pasteurized or unpasteurized hSA preparation as described herein has a CIE-inhibitor activity of at least 0.0075 IU per kg total protein. In certain embodiments, a pasteurized or unpasteurized hSA preparation as described herein has a serine protease inhibitor activity of at least 0.01 IU per kg total protein. In certain embodiments, a pasteurized or unpasteurized hSA preparation as described herein has a C1E-inhibitor activity of at least 0.01 IU per kg total protein. In certain embodiments, a pasteurized or unpasteurized hSA preparation as described herein has a serine protease inhibitor activity of at least 0.0125 IU per kg total protein. In certain embodiments, a pasteurized or unpasteurized hSA preparation as described herein has a CIE-inhibitor activity of at least 0.0125 IU per kg total protein. In certain embodiments, a pasteurized or unpasteurized hSA preparation as described herein has a serine protease inhibitor activity of at least 0.015 IU per kg total protein. In certain embodiments, a pasteurized or unpasteurized hSA preparation as described herein has a C1E-inhibitor activity of at least 0.015 IU per kg total protein. In certain embodiments, a pasteurized or unpasteurized hSA preparation as described herein has a serine protease inhibitor activity of 0.0025 to 0.025 IU per kg total protein. In certain embodiments, a pasteurized or unpasteurized hSA preparation as described herein has a C1E-inhibitor activity of 0.0025 to 0.025 IU per kg total protein. In certain embodiments, a pasteurized or unpasteurized hSA preparation as described herein has a serine protease inhibitor activity of 0.0025 to 0.02 IU per kg total protein. In certain embodiments, a pasteurized or unpasteurized hSA preparation as described herein has a CIE-inhibitor activity of 0.0025 to 0.02 IU per kg total protein. In certain embodiments, a pasteurized or unpasteurized hSA preparation as described herein has a serine protease inhibitor activity of 0.003 to 0.015 IU per kg total protein. In certain embodiments, a pasteurized or unpasteurized hSA preparation as described herein has a CIE-inhibitor activity of 0.003 to 0.015 IU per kg total protein. In certain embodiments, a pasteurized or unpasteurized hSA preparation as described herein has a serine protease inhibitor activity of 0.004 to 0.01 IU per kg total protein. In certain embodiments, a pasteurized or unpasteurized hSA preparation as described herein has a C1E-inhibitor activity of 0.004 to 0.01 IU per kg total protein.

As noted above, an hSA preparation as described herein, such as an hSA preparation having an albumin content of at least 5% w/v, such as 5% or 25% w/v, additionally or alternatively may have a PKA activity of less than or equal to 35 IU/ml after pasteurization. In certain embodiments, an hSA preparation as described herein, such as an hSA preparation having an albumin content of at least 5% w/v, such as 5% or 25% w/v, has a PKA activity of less than or equal to 35 IU/ml total protein after pasteurization and storage for 1 month or longer, including 3 months or longer, including 6 months and 12 months, at a temperature of 0-32° C., such as 18-25° C. In certain embodiments, an hSA preparation as described herein, such as an hSA preparation having an albumin content of at least 5% w/v, such as 5% or 25% w/v, has a PKA activity of less than or equal to 20 IU/ml after pasteurization. In certain embodiments, an hSA preparation as described herein, such as an hSA preparation having an albumin content of at least 5% w/v, such as 5% or 25% w/v, has a PKA activity of less than or equal to 20 IU/ml after pasteurization and storage for 1 month or longer, including 3 months or longer, including 6 months and 12 months, at a temperature of 18-25° C. In certain embodiments, an hSA preparation as described herein, such as an hSA preparation having an albumin content of at least 5% w/v, such as 5% or 25% w/v, has a PKA activity of less than or equal to 10 IU/ml after pasteurization. In certain embodiments, an hSA preparation as described herein, such as an hSA preparation having an albumin content of at least 5% w/v, such as 5% or 25% w/v, has a PKA activity of less than or equal to 10 IU/ml after pasteurization and storage for 1 month or longer, including 3 months or longer, including 6 months and 12 months, at a temperature of 18-25° C. In certain embodiments, an hSA preparation as described herein, such as an hSA preparation having an albumin content of at least 5% w/v, such as 5% or 25% w/v, has a PKA activity of less than or equal to 5 IU/ml after pasteurization. In certain embodiments, an hSA preparation as described herein, such as an hSA preparation having an albumin content of at least 5% w/v, such as 5% or 25% w/v, has a PKA activity of less than or equal to 5 IU/ml after pasteurization and storage for 1 month or longer, including 3 months or longer, including 6 months and 12 months, at a temperature of 18-25° C.

In some embodiments, an hSA preparation is “stable” when the preparation is one or both of physically and chemically stable over a storage period, for example, as may be determined according to the European Pharmacopoeia version 10.6. For example, an hSA preparation may be considered to be “stable” when its initial serine protease activity does not increase by more than 20 percent, by more than 10 percent, or by more than 5 percent when stored over a period of time, such as a period of time of 2 weeks or longer, such as 1 month, 3 months, or 6 months, at a storage temperature of 0-32° C., such as 18-25° C. In certain embodiments, the initial protease activity of a pasteurized hSA preparation as described herein does not increase by more than 20 percent when stored for 1 month at a storage temperature of 0-32° C. In certain embodiments, the initial protease activity of a pasteurized hSA preparation as described herein does not increase by more than 20 percent when stored for 1 month at a storage temperature of 18-25° C. In certain embodiments, the initial protease activity of a pasteurized hSA preparation as described herein does not increase by more than 20 percent when stored for 3 months at a storage temperature of 18-25° C. In certain embodiments, the initial protease activity of a pasteurized hSA preparation as described herein does not increase by more than 20 percent when stored for 6 months at a storage temperature of 18-25° C.

An hSA preparation as described herein may be frozen for storage prior to further processing or use. A suitable storage temperature can be between-20 and 37° C. In certain embodiments, the storage temperature is between 0 and 32° C. In certain embodiments, the storage temperature is between 2° and 32° C. In certain embodiments, the storage temperature is between 0 and 8° C. In certain embodiments, the storage temperature is 30-34° C. In certain embodiments, the storage temperature is 18-25° C. In certain embodiments, the storage temperature is 4-8° C. In certain embodiments, the storage temperature is 0-4° C. In certain embodiments, the storage temperature is below 32° C.

An hSA preparation as described herein may be formulated into an hSA drug product or pharmaceutical composition containing an appropriate amount of albumin as an active ingredient, such as an appropriate amount according to pharmacopeia standards such as Ph. Eur. or USP. Typically, a pasteurized hSA preparation as described herein is used for preparation of an hSA drug product.

Thus, for example, an hSA drug product made from an hSA preparation as described herein may comprise 5 g, 40 g, 50 g, 100 g, 150 g, 200 g, 250 g, or more, of albumin per kg. In certain embodiments, the drug product comprises 200-300 g of albumin per kg. In certain embodiments, the preparation comprises 250 g of albumin per kg. In certain embodiments, the preparation comprises 50 g of albumin per kg.

The following excipients may typically be included in an hSA drug product: sodium N-acetyl-tryptophan (stabilizer); sodium caprylate (stabilizer); sodium chloride (tonicity agent); water for injections (solvent). The protein content may be adjusted as required to manufacture 4% w/v, 5% w/v, 20% w/v and 25% w/v Human Albumin Solution (hSA), or any desired strength. In certain embodiments, the drug product does not include any preservatives.

An hSA drug product made from an hSA preparation as described herein may comprise 0.1-0.2 M sodium, less than 150-250 μg/L aluminium, and less than 0.001-0.003 M potassium. As noted above, the product may be stabilized with 0.01-0.03 M sodium N-acetyltryptophanate and/or 0.01-0.03 M sodium caprylate. In certain embodiments, the drug product contains 0.14 M sodium, less than 200 μg/L aluminium, and less than 0.002 M potassium. Such embodiments may be stabilized with 0.02 M sodium N-acetyltryptophanate and/or 0.02 M sodium caprylate.

In particular embodiments, an hSA drug product made from an hSA preparation as described herein has the following contents (for 4% w/v, 5% w/v, 20% w/v and 25% w/v hSA solutions, respectively):(i) Protein (4% w/v), sodium (140 mM) and caprylate (6.4 mM); (ii) Protein (5% w/v), sodium (140 mM) and caprylate (8 mM); (iii) Protein (20% w/v) and caprylate (32 mM); (iv) Protein (25% w/v) and caprylate (40 mM).

In further embodiments, an hSA drug product made from an hSA preparation as described herein has the following contents (for 4% w/v, 5% w/v, 20% w/v and 25% w/v hSA solutions, respectively): (i) Protein (4% w/v), 3.2 mM sodium N-acetyltryptophanate and 3.2 mM sodium caprylate; (ii) Protein (5% w/v), 4 mM sodium N-acetyltryptophanate and 4 mM sodium caprylate; (iii) Protein (20% w/v) 0.016 M sodium N-acetyltryptophanate and 0.016 M sodium caprylate; (iv) Protein (25% w/v) 0.02 M sodium N-acetyltryptophanate and 0.02 M sodium caprylate.

An hSA drug product made from an hSA preparation as described herein will preferably meet the appropriate pharmacopoeia standards. For example, following the test procedures described for a human albumin solution in the European Pharmacopoeia, an hSA preparation should be sterile; pyrogen-free; have endotoxin levels below 0.5 IU per mL for solutions having a protein content of less than 50 g/L, or less than 1.3 IU per mL for solutions having a protein content from 50 g/L to 200 g/L, or less than 1.7 IU/mL for solutions having a protein content of greater than 200 g/L; have an aluminium content of a maximum of 200 μg/L; have a prekallikrein activator (PKA) maximum content of 35 IU/mL; have a haem content such that absorbance of a test solution measured at 403 nm using water R as the compensation liquid is not greater than 0.15; have a potassium maximum content of 0.05 mmol per gram of protein; have a sodium maximum content of 160 mmol/L and 95% to 105% of the content of Na stated on the label; have a maximum content of 10% polymers and aggregates; not more than 5% of protein having a mobility different from the principal band by zone electrophoresis; having a pH of 6.7 to 7.3, and having a total protein content of not less than 9% and not more than 10% of the stated content on the label.

### EXAMPLES

### Analytical Methods

### Serine Protease Activity

Serine protease activity may be measured by standard assays that are known in the art, e.g. using commercially available chromogenic substrates. An example of a suitable assay is an assay using the chromogenic substrate S-2288 (H-D-Ile-Pro-Arg-pNA *2HCl, available e.g. from Diapharma). Cleavage of S-2288 by serine proteases with arginine specificity gives rise to the production of p-nitroaniline (pNA). The proteolytic activity is thus determined by pNA release. The formation of pNA can be followed spectrophotometrically at 405 nm, e.g. over a suitable time period. Suitable assays may be based on those described in Friberger, Scand. J. Clin. Lab. Inves.t Suppl. (1982) 162:1-298. The rate of formation of pNA can be compared to suitable standards having a known amount of proteolytic activity.

### PKA Activity

PKA activity can be measured by standard assays that are known in the art, e.g. using commercially available chromogenic substrates. An example of a suitable assay is an assay using the chromogenic S-2302™ (Chromogenix) (H-D-Pro-Phe-Arg-p-nitroaniline dihydrochloride, available, e.g., from Diapharma), which indirectly measures PKA activity by assessing kallikrein-like activity. Cleavage of S-2302 by kallikrein-like activity gives rise to the production of p-nitroaniline (pNA). The proteolytic activity is thus determined by pNA release. The formation of pNA can be followed spectrophotometrically at 405 nm, e.g., over a suitable time period. The rate of formation of pNA can be compared to suitable standards having a known amount of proteolytic activity.

### Evaluation of CIE-Inhibitor Activity

C1E-inhibitor activity was assessed by radial immunodiffusion. For this assay, holes are pierced in an agarose gel with antiserum (e.g., an antibody specific to CIE-inhibitor). A small amount of sample is pipetted into the hole and diffuses into the gel. A precipitation ring is formed at the point where the ratio of antigen (in the sample) and antibody (in the gel) is ideal for immunoprecipitation. The precipitated immune complexes are insoluble in aqueous solutions at pH 7 and immobilised in the gel. After washing out the soluble proteins from the gel, the precipitation rings are permanently stained. The areas enclosed by the precipitation circles are a measure of the antigen activity. The samples can be quantified using a calibration curve from a plasma pool.

### Example 1: Preparation of hSA Preparations

This example illustrates the preparation of an hSA preparation as described herein. Although illustrated with the use of cryo-poor plasma as the fraction subjected to extraction, it should be understood that the extraction can be made at any step of the fractionation process.

Cryo-poor plasma is subjected to fractionation to obtain Fraction V precipitate by a process that does not include an extraction of C1E-inhibitor, to obtain unadsorbed material (also referred to herein as a “first albumin-containing fraction”) comprising Fraction V precipitate.

Cryo-poor plasma from the same or different source/batch used to obtain the first albumin-containing fraction is subjected to extraction as described herein, using two anion exchange chromatography steps, to obtain an adsorbed material (also referred to herein as a “second albumin-containing fraction”). The two anion exchange chromatography steps include a chromatography step using a DEAE resin (to remove PTC) and a chromatography step using a QAE resin (to remove C1E-inhibitor) The adsorbed material is subjected to fractionation to obtain Fraction V precipitate.

The first (unadsorbed, C1E-inhibitor containing) and second (adsorbed, C1E-inhibitor depleted) fractions of Fraction V precipitate are mixed in a desired weight:weight ratio, such as about 25:75 or about 33:67, or other weight:weight mixing ratios as disclosed herein and illustrated below. The ratio can be selected to maintain a desired level of CIE-inhibitor activity to inhibit protease activation in the preparation.

### Example 2: Protease Activity in Preparations Prepared with Different Mixing Ratios

For this experiment, unadsorbed and adsorbed Fraction V precipitates were prepared as described above, mixed at different mixing ratios (weight:weight), and assessed for protease activity. Samples were prepared for testing by the procedures outlined below.

### Preparation of Samples:

Fraction V precipitates prepared as described above were allowed to temper at −80° C. for a minimum of 24 hours and then mixed at different weight:weight ratios (0:100, 99:1, 10:90, 25:75, 33:67, 100:0) For each mixing ratio, 1 kg of mixed Fraction V precipitate was re-suspended with water for injection (WFI) at a targeted temperature of 0° C. After mixing for 75 minutes, samples were taken for intermediate testing. The pH was checked to be in a range of 4.6 to 4.8 and adjusted with 1 M NaOH or 1 M HCl, if necessary. The suspensions were then mixed for an additional period of 2 hours at a targeted temperature of 0° C.

Filter aid was added to the suspensions and mixed.

The suspensions were recirculated through a filter press until turbidity was lower than 20 NTU. Filtrate was collected, and samples were then taken for intermediate testing. The pH was adjusted to be in a range of 7.2 to 7.3.

The filtrate was filtered through a 0.2 μm bottle-top filter, and protein concentration was determined by A280 analysis. The filtrate was concentrated to a target protein concentration of 13.5% w/v using a 10 kD molecular weight cut off (MWCO) membrane. The concentrate was then diafiltered into 1.8% NaCl followed by WFI. Samples were then taken for intermediate testing. Protein concentration was again determined by A280 analysis, and the 13.5% concentrate was split. One portion was further concentrated to 29.2% to prepare a 25% w/v protein formulation (referred to as “25% bulk” in some figures), and another portion was diluted to prepare a 5% w/v protein formulation (referred to as “5% bulk” in some figures).

To prepare the 5% protein formulation, 13.5% concentrate was diluted to 5.44% using WFI. Stabilizer buffer (sodium tryptophan and sodium caprylate) was added at a volume equal to 0.44% of the bulk weight. NaCl was added to reach a final concentration of 130 mM and mixed for 15 minutes. Samples were taken for in-process testing. WFI was added to reach a target density in a range of 1.0167-1.0177 g/ml. The pH was adjusted to be in a range of 6.4 to 7.4 using either 1 M NaOH or 1M HCl. The 5% formulation was then filtered through a 0.2 μm bottle-top filter. Samples were taken for in-process testing.

To prepare the 25% protein formulation, the 29.2% concentrate was diluted to 27.2% using WFI. Stabilizer buffer (as above) was added at a volume equal to 2.2% of the bulk weight. NaCl was added to reach a final concentration of 60 mM and mixed for 15 minutes. Samples were taken for in-process testing. WFI was added to reach a final target density of 1.0696 g/mL. The pH was adjusted to be in a range of 6.4 to 7.4 using either 1M NaOH or 1M HCl. The 25% bulk was then filtered through a 0.2 μm bottle-top filter. Samples were taken for in-process testing.

Pre-washed vials were filled with either the 5% or the 25% protein preparations, and the vials were stoppered, capped, and crimped. The vials were pasteurized for between 10 and 11 hours at 60±0.5° C. in a water bath. Vials were then removed and samples were taken for container testing. Remaining vials were kept in the dark at ambient temperature for stability testing at 1, 3, and 6-month time points, as reported in Example 3 below.

### Protease Activity Levels:

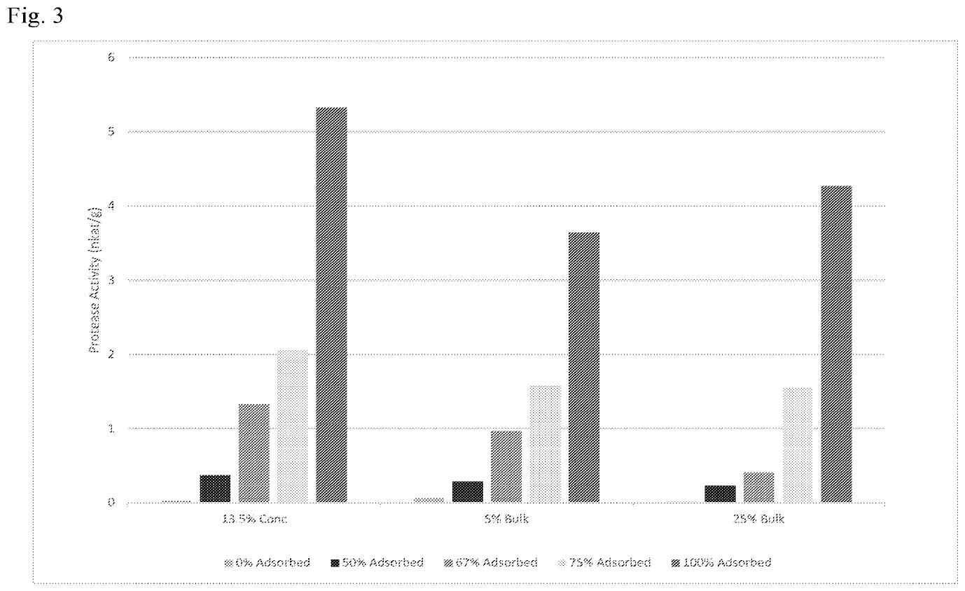

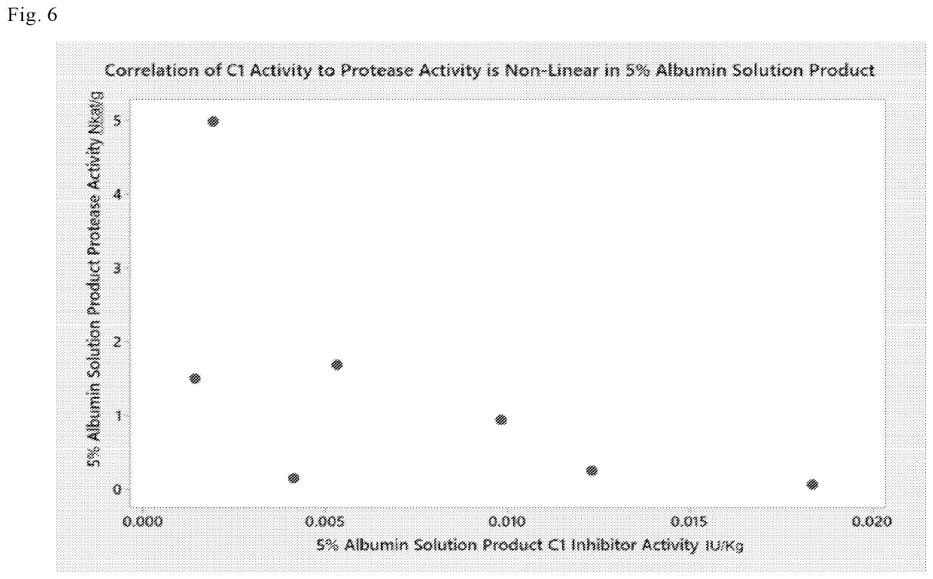

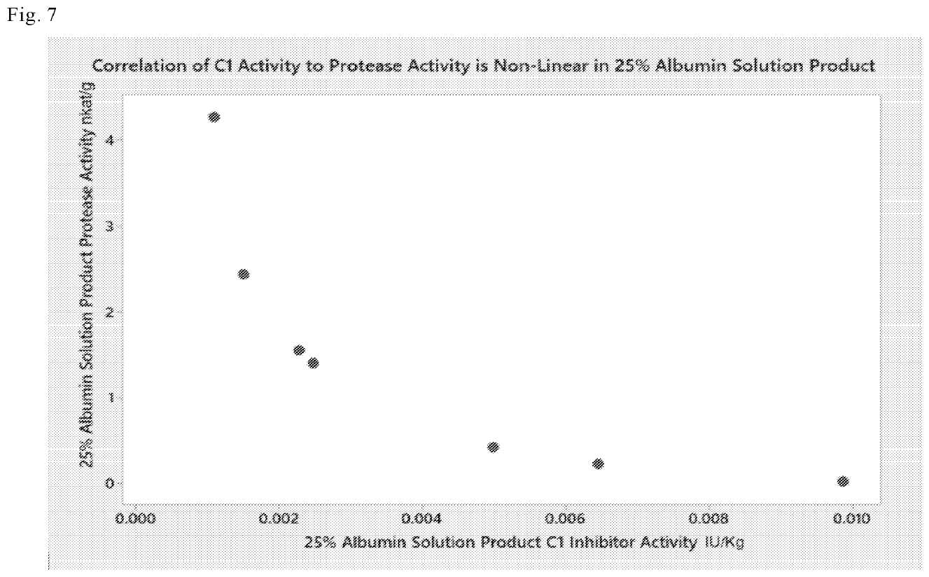

Serine protease activity levels (FIGS. 3-5) and C1E-inhibitor activity levels (FIGS. 6-10) of the preparations described above were measured. As seen in FIGS. 6 and 7, serine protease activity levels and C1E-inhibitor activity levels were found to be inversely correlated.

FIG. 3 reports the serine-like protease activity (nkat/g total protein) of unpasteurized Fraction V precipitate hSA preparations prepared using various weight:weight ratios of first (unadsorbed) and second (adsorbed) albumin-containing fractions of as described above: 0% adsorbed=100:0; 50% adsorbed=50:50, 67% adsorbed=33:67; 75% adsorbed=25:75; 100% adsorbed=0:100. As noted above, after preparing the mixed Fraction V precipitates, the preparations were prepared at different protein concentrations (13.5%, 5% or 25% w/v albumin). As noted above, serine-like protease activity was determined using the chromogenic substrate S-2288™ (Chromogenix) (H-D-Isoleucyl-L-prolyl-L-arginine-p-nitroaniline dihydrochloride) which is sensitive to a broad spectrum of serine proteases.

The results show that the serine protease activity is nearly undetectable in the 0% adsorbed samples, and that serine protease activity increases with increasing relative amount of adsorbed fraction (second albumin-containing fraction), although serine protease activity does not exhibit a linear relationship with mixing ratio. This indicates that combining the first (unadsorbed, protease inhibitor-containing) fraction with the second (adsorbed, protease inhibitor-depleted) fraction has a net effect of inhibiting protease activation as opposed to merely a dilution effect. This phenomenon is further supported by the non-linear correlation between protease activity and C1E-inhibitor activity seen in FIGS. 6 and 7 discussed below.

The serine protease activity of samples subjected to pasteurization (not shown) was nearly undetectable for all samples, indicating that pasteurization eliminates uninhibited proteolytic activity down to a certain threshold.

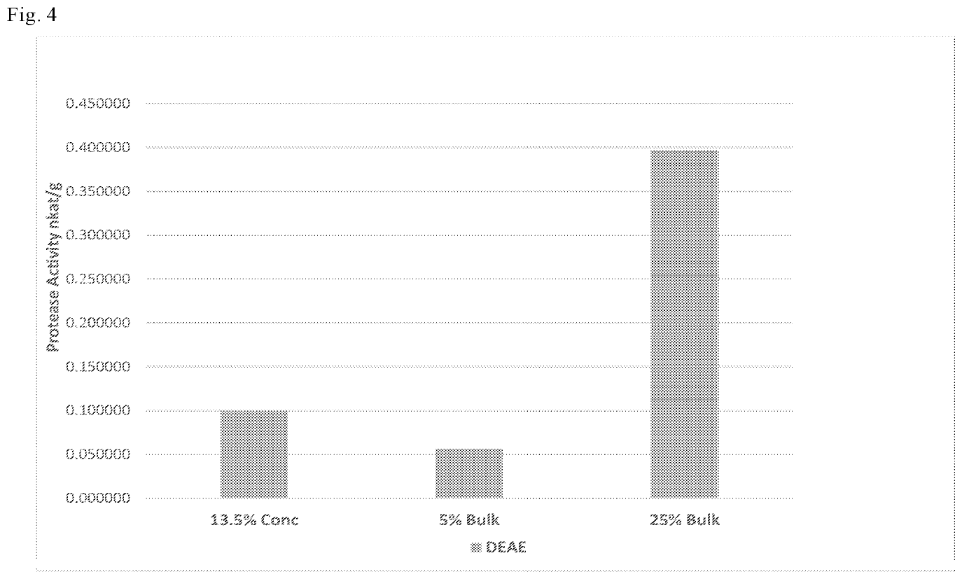

FIG. 4 reports serine protease activity (nkat/g total protein) in unpasteurized Fraction V precipitates obtained as described above, except the adsorbed cryo-poor plasma only was subjected to adsorption on a diethyl aminoethyl (DEAE) resin (not also a QAEE resin), and then fractionated to obtain Fraction V precipitate, and then prepared at different protein concentrations (13.5%, 5% or 25% w/v albumin). The results show that the serine protease activity was very low in all samples, indicating that the DEAE adsorption was not effective to remove all C1E-inhibitor (or other serine protease inhibitor). (Note the y-axes scale is different for FIG. 3 and FIG. 4).

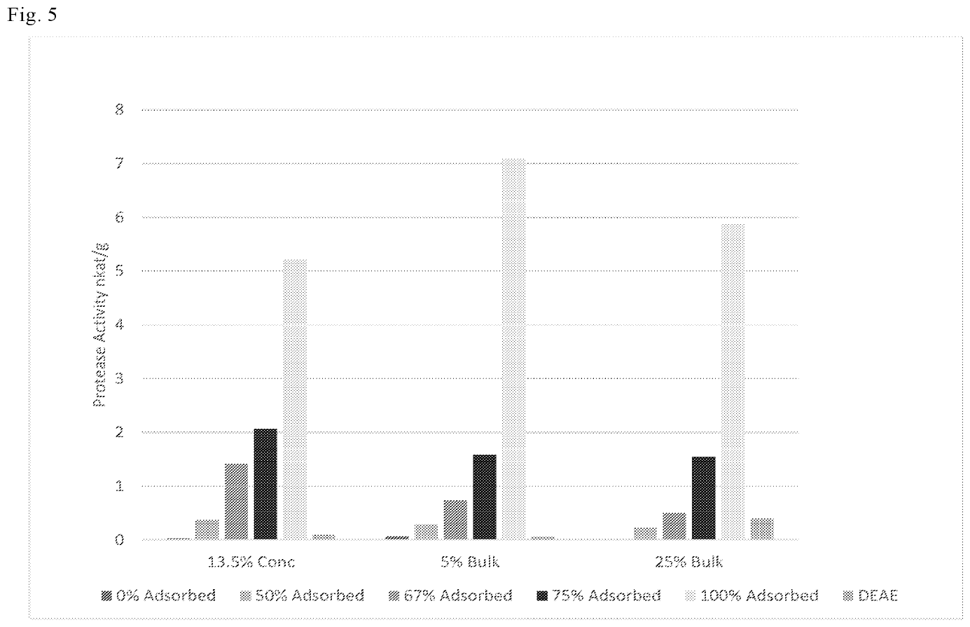

FIG. 5 reports serine protease activity (nkat/g total protein) in additional unpasteurized Fraction V precipitate hSA preparations prepared as described for FIG. 3 and FIG. 4, with data reported on the same scale.

FIG. 6 and FIG. 7 illustrate the non-linear correlation of C1E-inhibitor activity (IU/kg total protein) to serine protease activity (nkat/g total protein) in unpasteurized 5% and 25% w/v albumin preparations prepared as described for FIG. 3. The data indicate that a very small amount of C1E-inhibitor activity (e.g., less than 0.004 nkat/kg protein as seen in FIG. 7) can lead to a significant reduction of serine protease activity.

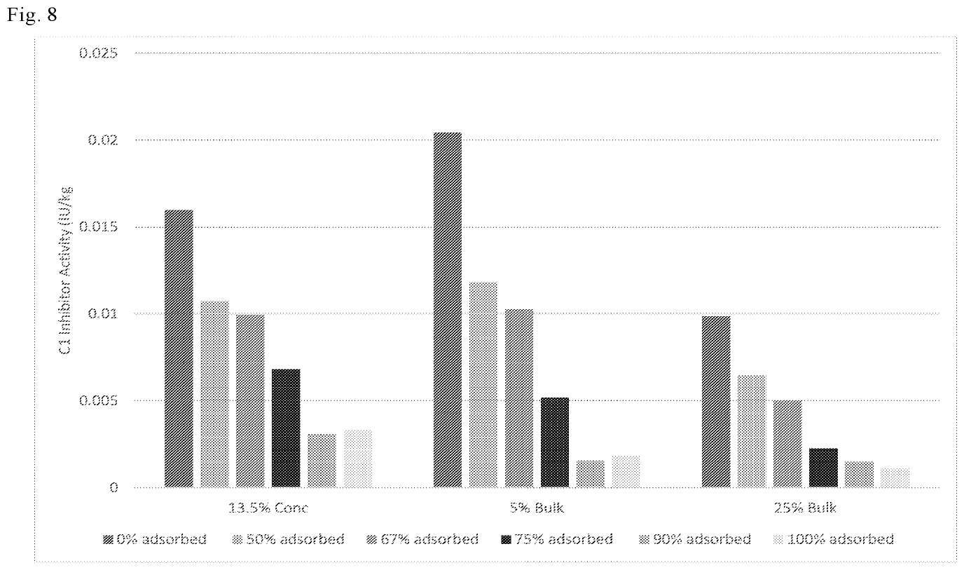

FIG. 8 reports C1E-inhibitor activity (IU/kg total protein) in unpasteurized Fraction V precipitate hSA preparations prepared as described above for FIG. 3 with different mixing ratios (0% adsorbed=100:0; 50% adsorbed=50:50, 67% adsorbed=33:67; 75% adsorbed=25:75; 90% adsorbed=10:90; 100% adsorbed=0:100), and then prepared at different protein concentrations (13.5%, 5% or 25% w/v albumin). The C1E-inhibitor activity data exhibits an opposite trend compared to the serine protease activity data discussed above, with CE1-inhibitor activity decreasing with increasing relative amount of adsorbed fraction (second albumin-containing fraction). These results confirm that the QAE adsorption removes CE1-inhibitor activity, although even the 100% adsorbed samples exhibited a small level of CE1-inhibitor activity.

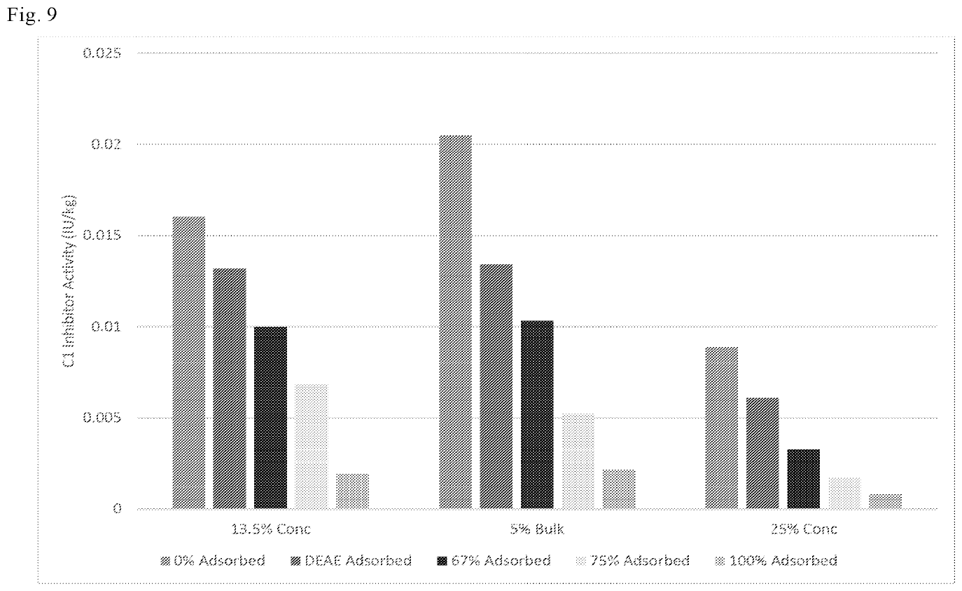

FIG. 9 reports C1E-inhibitor activity (IU/kg total protein) in unpasteurized Fraction V precipitate hSA preparations prepared as described for FIG. 3 with different mixing ratios (0% adsorbed=100:0; 67% adsorbed=33:67; 75% adsorbed=25:75; 100% adsorbed=0:100), and then prepared at different protein concentrations (13.5%, 5% or 25% w/v albumin), and also reports C1E-inhibitor activity of samples prepared as described for FIG. 4 (“DEAE adsorbed”). Comparing the results for the 0% adsorbed samples with those for the DEAE adsorbed samples indicates that the DEAE adsorption removes some C1E-inhibitor.

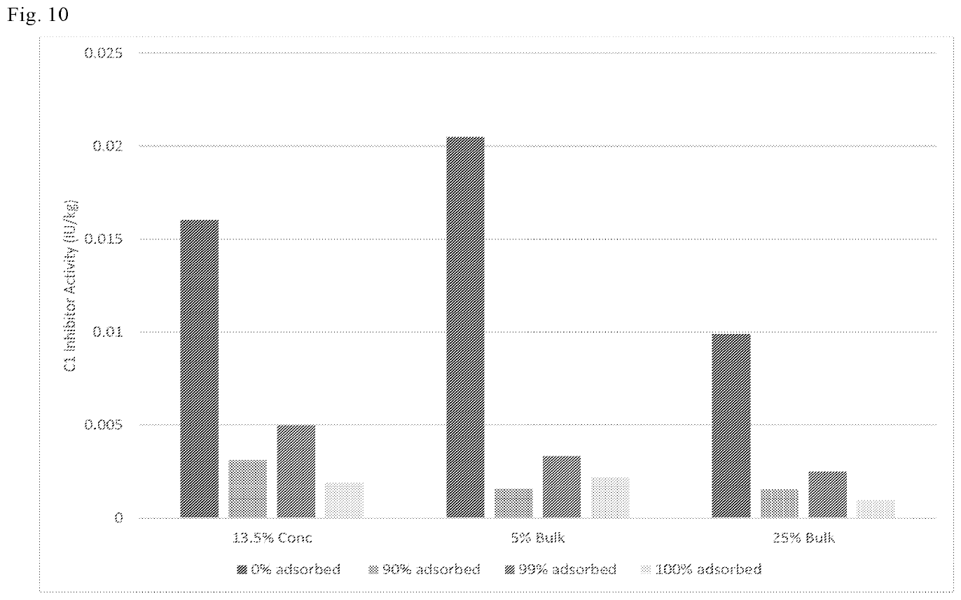

FIG. 10 compares C1E-inhibitor activity (IU/kg total protein) in unpasteurized Fraction V precipitate hSA preparations prepared as described for FIG. 3 using different mixing ratios (0% adsorbed=100:0; 90% adsorbed=10:90; 99% adsorbed=1:99; 100% adsorbed=0:100), and then prepared at different protein concentrations (13.5%, 5% or 25% w/v albumin).

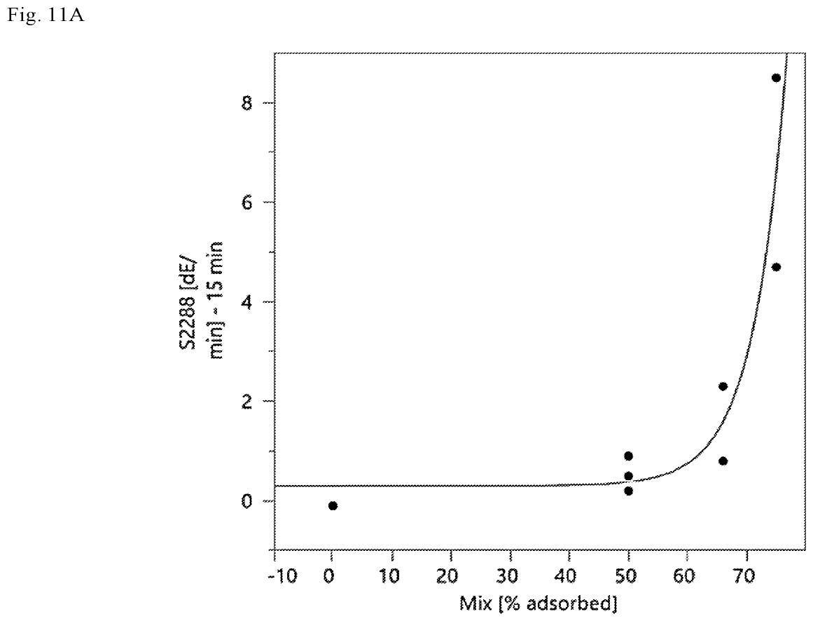

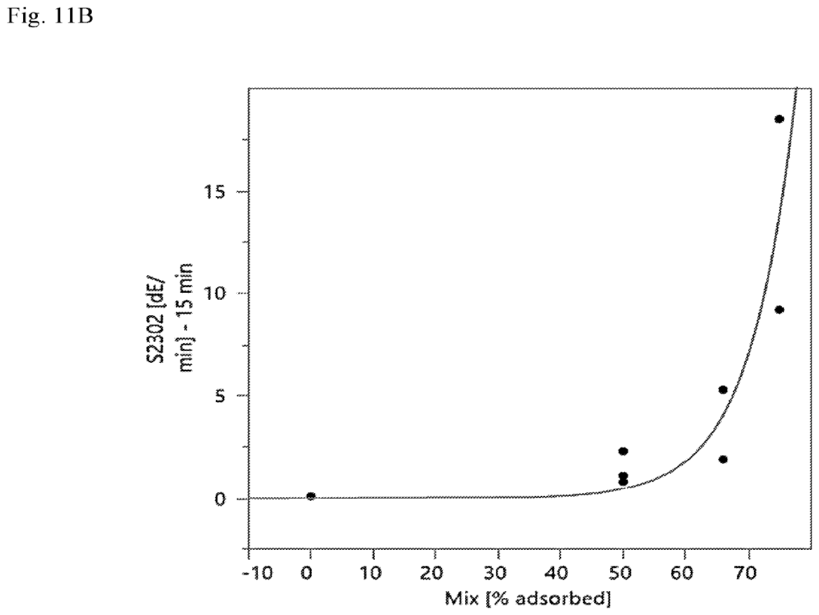

FIG. 11A and FIG. 11B report the serine-like protease activity (FIG. 11A) determined using the chromogenic substrate S-2288™ (Chromogenix) and PKA activity (FIG. 11B) determined using the chromogenic substrate S-2302™ (Chromogenix) (H-D-Prolyl-L-phenylalanyl-L-arginine-p-nitroaniline dihydrochloride) in unpasteurized 25% albumin preparations prepared from Fraction V precipitate hSA preparations as described above for FIG. 3, at different mixing ratios (0=100:0; 50=50:50; 67=33:67; 75=25:75). The results are consistent with the results reported in FIG. 3, and show that PKA activity increases in a non-linear (exponential) manner with increasing relative amount of adsorbed fraction (second albumin-containing fraction).

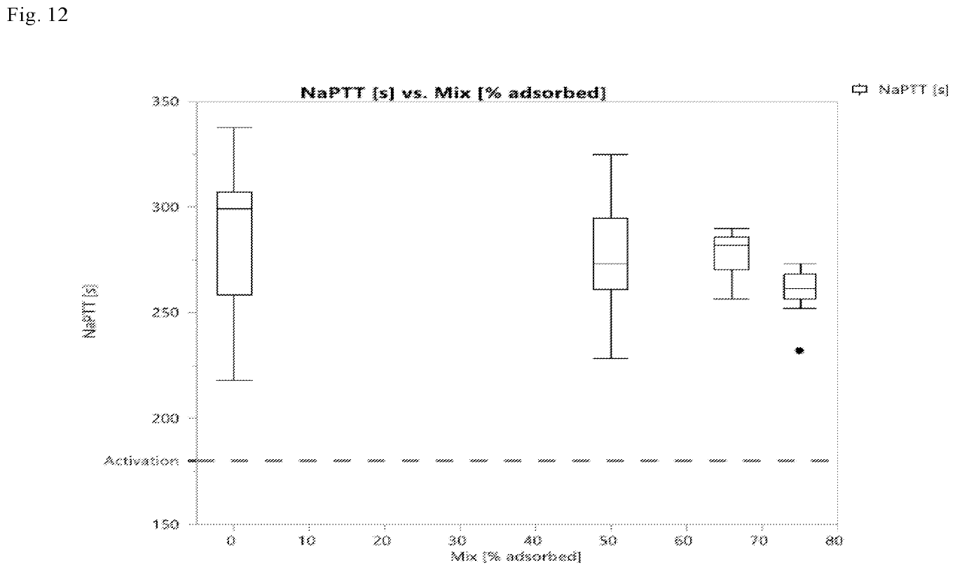

FIG. 12 reports the non-activated partial thromboplastin time (NaPTT) in unpasteurized 25% albumin preparations prepared as described for FIGS. 11A-B. NaPTT is a coagulation assay sensitive to the presence of activated coagulation factors. For testing, samples were diluted 1/10 and 1/100 in buffer solution. Human normal plasma was mixed with rabbit brain cephalin and incubated at 37° C. The diluted samples and CaCl₂) were added, and clotting time was measured. A buffer solution was included in the same run of tests as a control. Results are expressed as means of duplicates, in seconds, for each dilution assayed. The test was considered valid when the coagulation time for the control tube was 200-320 seconds and the time for the samples yielded results longer than 150 seconds. FIG. 12 is a box-and-whisker plot of the obtained data, graphically depicting data in quartiles.

### Example 3: Stability of hSA Preparations

As noted above, pasteurized samples prepared as described in Example 2 were kept in the dark (to avoid discoloration) at ambient temperature (18-25° C.) for stability testing at 1, 3, and 6-month time points. Pasteurized samples prepared as described for FIG. 4 (e.g., with only DEAE adsorption) also were stored under the same conditions. Results are reported in Table 1 below, and demonstrate the stability of hSA preparations as described herein with regard to maintaining a low proteolytic activity after pasteurization and storage. The 5% w/v protein formulation prepared from a 10:90 mixing ratio (90% adsorbed fraction) was the only sample to have detectable proteolytic activity at the 1-month time point.

The results indicate that pasteurized compositions prepared with mixing ratios of up to 75% adsorbed fraction remain stable with serine protease activity less than 0.06 nkat/g protein for at least 3 months.

TABLE 1
Proteolytic activity (nkat/g protein) in
final container (after pasteurization)

| Sample | | Initial | Activity | Activity |
|---|---|---|---|---|
| (w/v | Mixing Ratio | Activity | at 1 Mo. | at 3 Mo. |
| protein) | (% Adsorbed) | (Avg.) | (Avg.) | (Avg.) |
| 5% | 0% | <0.06 | <0.06 | <0.06 |
| 25% | | <0.01 | <0.01* | <0.01 |
| 5% | 50% | <0.06 | <0.06 | <0.06 |
| 25% | | <0.01 | <0.01 | <0.01 |
| 5% | 66% | 0.08 | <0.06 | <0.06 |
| 25% | | <0.01 | <0.01 | <0.01 |
| 5% | 75% | <0.06 | <0.06 | <0.06 |
| 25% | | <0.01 | <0.01 | <0.01 |
| 5% | 90% | <0.06 | 0.12 | 0.18 |
| 25% | | <0.01 | <0.01 | <0.01 |
| 5% | 99% | <0.06 | <0.06 | 0.06 |
| 25% | | <0.01 | <0.01 | 0.03 |
| 5% | 100% | 0.16 | 0.50 | 0.64 |
| 25% | | 0.02** | 0.07 | 0.09 |
| 5% | 100% DEAE | <0.06 | <0.06 | <0.06 |
| 25% | Adsorbed Only | <0.01 | <0.01 | <0.01 |

n = 3 unless otherwise stated
*n = 2 for this control
**two samples were below the limit of detection and one had detectable proteolytic activity

Additional stability data for the 5% w/v and 25% w/v protein formulations (after pasteurization and storage under the same conditions for 1 month, 3 month, and 6 months) are reported in Tables 2 and 3 below.

TABLE 2
Proteolytic activity (nkat/g total protein) in 5% w/v
product in final container (after pasteurization)

| | | Initial | | | |
|---|---|---|---|---|---|
| | | Activity | 1 M | 3 M | 6 M |
| | Total | nkat/g | nkat/g | nkat/g | nkat/g |
| | Protein | total | total | total | total |
| Adsorption | (g/kg) | protein | protein | protein | protein |
| 0% | 49.5 | 0.06 | 0.06 | 0.06 | N/A |
| | 49.1 | 0.06 | 0.06 | 0.06 | 0.06 |
| | 51.3 | 0.06 | 0.06 | 0.06 | 0.06 |
| Average: | | 0.06 | 0.06 | 0.06 | 0.06 |
| 50% | 48.8 | 0.06 | 0.06 | 0.06 | 0.06 |
| | 50.2 | 0.06 | 0.06 | 0.06 | 0.06 |
| | 52.6 | 0.06 | 0.06 | 0.06 | N/A |
| Average: | | 0.06 | 0.06 | 0.06 | 0.06 |
| 67% | 49.9 | 0.06 | 0.06 | 0.06 | 0.06 |
| | 50.7 | 0.12 | 0.06 | 0.06 | 0.06 |
| | 51.3 | 0.06 | 0.06 | 0.06 | 0.06 |
| Average: | | 0.08 | 0.06 | 0.06 | 0.06 |
| 75% | 49.9 | 0.06 | 0.06 | 0.06 | 0.06 |
| | 50.6 | 0.06 | 0.06 | 0.06 | 0.06 |
| | 52 | 0.06 | 0.06 | 0.06 | 0.06 |
| Average: | | 0.06 | 0.06 | 0.06 | 0.06 |
| 90% | 49.8 | 0.06 | 0.12 | 0.18 | N/A |
| 99% | 52.4 | 0.06 | 0.06 | 0.06 | N/A |
| 100% | 50.2 | 0.20 | 0.80 | 1.04 | N/A |
| | 51.3 | 0.14 | 0.47 | 0.60 | 0.96 |
| | 44.9 | 0.13 | 0.24 | 0.27 | 0.31 |
| Average: | | 0.16 | 0.50 | 0.64 | 0.63 |
| DEAE Only | 53.1 | 0.06 | 0.06 | 0.06 | N/A |

TABLE 3
Proteolytic activity (nkat/g total protein)] in
25% w/v product in final container (after pasteurization)

| | | t = 0 | 1 M | 3 M | 6 M |
|---|---|---|---|---|---|
| | Total | nkat/g | nkat/g | nkat/g | nkat/g |
| | Protein | total | total | total | total |
| Adsorption | (g/kg) | protein | protein | protein | protein |
| 0% | 251 | 0.01 | 0.01 | 0.01 | 0.01 |
| | 255.5 | 0.01 | 0.01 | 0.01 | 0.01 |
| | 259.4 | 0.01 | 0.01 | 0.01 | 0.01 |
| Average: | | 0.01 | 0.01 | 0.01 | 0.01 |
| 50% | 254.9 | 0.01 | 0.01 | 0.01 | 0.01 |
| | 244.3 | 0.01 | 0.01 | 0.01 | 0.01 |
| | 256 | 0.01 | 0.01 | 0.01 | N/A |
| Average: | | 0.01 | 0.01 | 0.01 | 0.01 |
| 67% | N/A | N/A | N/A | N/A | N/A |
| | 254 | 0.01 | 0.01 | 0.01 | 0.01 |
| | 256.4 | 0.01 | 0.01 | 0.01 | 0.01 |
| Average: | | 0.01 | 0.01 | 0.01 | 0.01 |
| 75% | 254.3 | 0.01 | 0.01 | 0.01 | 0.01 |
| | 255.4 | 0.01 | 0.01 | 0.01 | 0.01 |
| | 256.8 | 0.01 | 0.01 | 0.01 | 0.01 |
| Average: | | 0.01 | 0.01 | 0.01 | 0.01 |
| 90% | 261.1 | 0.01 | 0.01 | 0.01 | N/A |
| 99% | 252.3 | 0.01 | 0.01 | 0.03 | N/A |
| 100% | 253.8 | 0.03 | 0.09 | 0.11 | 0.10 |
| | 248.1 | 0.03 | 0.10 | 0.15 | 0.18 |
| | 309.66 | 0.01 | 0.01 | 0.01 | N/A |
| Average: | | 0.02 | 0.07 | 0.09 | 0.14 |
| DEAE Only | 256.8 | 0.01 | 0.01 | 0.01 | N/A |

All final container samples and stability samples met final container requirements in all testing completed. This indicates that proteolytic activity is successfully inhibited in the samples, which were prepared at different mixing ratios.

5% and 25% hSA preparations were prepared as described above at pilot scale using a mixing ratio of 25:75 (75% adsorbed). All pilot scale final container samples met Pharmacopoeia requirements for a human albumin solution (including PKA activity of ≤35 IU/mL, with PKA activity being <5 IU/mL after pasteurization and storage under conditions outlined above for 1 month, 3 months, or 6 months for all preparations), which indicates that the methods described herein do not undermine critical product parameters.

### CONCLUSIONS

Taken together, the experimental data reported herein indicate that mixing a first (unadsorbed, protease inhibitor-containing) fraction with a second (adsorbed, protease inhibitor-depleted) fraction can provide sufficient C1E-inhibitor activity to inhibit protease activity and maintain protease activity in final products (e.g., pasteurized products) at less than detectable levels. For example, mixing ratios with 25% or more unadsorbed fraction, such as mixing ratios of about 25:75 (unadsorbed:adsorbed) or about 33:67 (unadsorbed:adsorbed), provide sufficient C1E-inhibitor activity for a storage-stable product.

As also explained above, the experimental data indicate that the effect is greater than what would be expected by a dilution effect. For example, the results reported in FIG. 8 show that there is more than a 25% difference in C1E-inhibitor activity between the preparation prepared with a 25:75 mixing ratio and the preparation prepared with a 0:100 mixing ratio. If the effect was simply due to dilution, changes in activity closer to 25% would have been expected. However, in the data reported in FIG. 8, there is a 34.6% decrease in C1E-inhibitor activity between the composition prepared with 75% adsorbed (C1E-inhibitor depleted) fraction and the composition prepared with 100% adsorbed (C1E-inhibitor depleted) fraction, for the 5% protein products. The difference seen for the 25% protein products is even more dramatic, at 47.8%. Comparing the results reported in FIG. 8 for the 50:50 and 0:100 mixing ratio compositions and the 50:50 and 25:75 mixing ratio compositions also reveals an effect greater than a dilution effect. This demonstrates that the addition of adsorbed (C1E-inhibitor depleted) fraction to unadsorbed (C1E-inhibitor containing) fraction is inhibiting C1E-inhibitor activity, rather than merely diluting the activity.

Although most C1E-inhibitor activity and protease activity is removed through pasteurization, it is notable that protease activity remains below detection after storage for products prepared using mixing ratios of 25% or more unadsorbed fraction. This is because even small amounts of serine protease activity remaining after pasteurization can trigger a protease cascade during storage. The stability of the preparations described herein suggests that the C1E-inhibitor present in the preparations (and, in some embodiments, other protease inhibitors present) effectively control (limit) serine protease activity throughout the manufacturing process, and also effectively control (limit) serine protease activity remaining after pasteurization.

1. A method of preparing a human serum albumin preparation, comprising extracting an amount of C1 esterase inhibitor (C1E-inhibitor) from an albumin-containing fraction of human plasma to obtain an albumin preparation comprising an amount of C1E-inhibitor effective to inhibit protease activity in the albumin preparation.

2. The method according to claim 1, wherein, the albumin preparation has an albumin content of at least 5% w/v, and, after pasteurization and storage at a temperature of 18-25° C. for 1 month, the albumin preparation has one or both of (i) a serine protease activity of less than or equal to 0.8 nkat/g total protein and (ii) a prekallikrein-activator (PKA) activity of less than or equal to 35 IU/ml total protein.

3. The method according to claim 1, wherein the method further comprises, in the same extracting step or separate extracting step(s), extracting from the albumin-containing fraction of human plasma one or more additional serine protease inhibitors selected from alpha1-proteinase-inhibitor, antithrombin III, alpha1-antichymotrypsin, alpha2-antiplasmin, alpha2-macroglobulin, inter-alpha-trypsin inhibitor and beta1-anticollagenase (collagenase).

4. The method according to claim 1, wherein the method comprises mixing a first albumin-containing fraction of human plasma with a second albumin-containing fraction of human plasma to obtain the albumin preparation, wherein the second albumin-containing fraction of human plasma is obtained by a process comprising extracting an amount of C1E-inhibitor from an albumin-containing fraction of human plasma, and wherein the first albumin-containing fraction of human plasma is not subjected to said extracting.

5. The method according to claim 4, wherein the method further comprises, prior to the mixing, extracting from the second albumin-containing fraction of human plasma one or more additional serine protease inhibitors selected from alpha1-proteinase-inhibitor, antithrombin III, alpha1-antichymotrypsin, alpha2-antiplasmin, alpha2-macroglobulin, inter-alpha-trypsin inhibitor and beta1-anticollagenase (collagenase).

6. The method according to claim 1, wherein the fraction of human plasma subjected to extracting is selected from cryo-poor plasma and an ethanol fraction of human plasma of a human plasma fractionation process.

7. (canceled)

8. The method according to claim 1, wherein the fraction of human plasma subjected to extracting is selected from:
Supernatant I of a Cohn/Oncley industrial plasma fractionation or an equivalent fraction of a different plasma fractionation process;
Supernatant II+III of a Cohn/Oncley industrial plasma fractionation or an equivalent fraction of a different plasma fractionation process;
Supernatant I+II+III of a Cohn/Oncley industrial plasma fractionation or an equivalent fraction of a different plasma fractionation process;
Fraction I+II+III+IV filtrate of a Cohn/Oncley industrial plasma fractionation or an equivalent fraction of a different plasma fractionation process;
Supernatant IV of a Cohn/Oncley industrial plasma fractionation or an equivalent fraction of a different plasma fractionation process;
Supernatant IV1 of a Cohn/Oncley industrial plasma fractionation or an equivalent fraction of a different plasma fractionation process;
Supernatant IV4 of a Cohn/Oncley industrial plasma fractionation or an equivalent fraction of a different plasma fractionation process; and
Fraction V precipitate of a Cohn/Oncley industrial plasma fractionation or an equivalent fraction of a different plasma fractionation process.

9. The method according to claim 1, wherein the fraction of human plasma subjected to extracting is selected from:
Supernatant A of a Kistler/Nitschmann industrial plasma fractionation;
Filtrate A of a Kistler/Nitschmann industrial plasma fractionation;
Fraction I+II+III suspension of a Kistler/Nitschmann industrial plasma fractionation;
Supernatant IV of a Kistler/Nitschmann industrial plasma fractionation; and
Precipitate C of a Kistler/Nitschmann industrial plasma fractionation.

10. The method according to claim 1, wherein the extracting comprises adsorption on a solid support, optionally wherein the adsorption on a solid support only partially removes one or more of C1E-inhibitor and one or more additional protease inhibitors present in the albumin-containing fraction subject to extracting.

11-12. (canceled)

13. The method according to claim 1, wherein the extracting comprises a chromatography step on a strong anion exchange resin, optionally wherein the strong anion exchange resin comprises quaternary amino ethyl (QAE) groups.

14. The method according to claim 1, wherein the method comprises, prior to the extracting, subjecting the fraction of human plasma subject to extracting to a chromatography step on a weak anion exchange resin to remove Prothrombin Complex (PTC), optionally wherein the weak anion exchange resin comprises diethyl aminoethyl (DEAE) groups.

15. The method according to claim 4, wherein the first and second albumin-containing fractions of human plasma subjected to mixing are filtrates of the same fraction(s) of the same plasma fractionation process, optionally wherein the first and second albumin-containing fractions of human plasma subjected to mixing are Fraction V precipitates.

16. (canceled)

17. The method according to claim 4, wherein the fraction of human plasma subjected to extracting is selected from:
cryo-poor plasma, wherein the method further comprises fractionating the extracted cryo-poor plasma to obtain Fraction V precipitate, wherein the first and second albumin-containing fractions of human plasma subjected to mixing are Fraction V precipitates;
Fraction V precipitate, optionally wherein the Fraction V precipitate is resuspended to obtain a solution or suspension prior to the extracting, and wherein the first and second albumin-containing fractions of human plasma subjected to mixing are Fraction V precipitates;
Fraction V suspension; and
Fraction C precipitate, optionally wherein the Fraction C precipitate is resuspended to obtain a solution or suspension prior to the extracting, and wherein the first and second albumin-containing fractions of human plasma subjected to mixing are Fraction C precipitates.

18-20. (canceled)

21. The method according to claim 4, wherein the weight:weight ratio of the first and second albumin-containing fractions of human plasma subjected to mixing is from 10:90 to 90:10.

22-23. (canceled)

24. The method according to claim 4, wherein the weight:weight ratio of the first and second albumin-containing fractions of human plasma subjected to mixing is from about 25:75 to about 33:67.

25-26. (canceled)

27. The method according to claim 1, further comprising subjecting the albumin preparation to pasteurization.

28. The method according to claim 4, wherein the method comprises:
mixing a first albumin-containing fraction of human plasma with a second albumin-containing fraction of human plasma at a weight:weight ratio of about 33:67 to obtain the albumin preparation, wherein the first and second albumin-containing fractions of human plasma are Fraction V precipitates of a Cohn/Oncley industrial plasma fractionation or equivalent fractions of a different plasma fractionation process, wherein:
the second albumin-containing fraction of human plasma is obtained by a process comprising (i) a chromatography step on a weak anion exchange resin to remove Prothrombin Complex (PTC), optionally wherein the weak anion exchange resin comprises DEAE groups; (ii) extracting an amount of C1E-inhibitor by a chromatography step on a strong anion exchange resin, optionally wherein the strong anion exchange resin comprises quaternary amino ethyl (QAE) groups, (iii) optionally, extracting one or more additional serine protease inhibitors selected from alpha1-proteinase-inhibitor, antithrombin III, alpha1-antichymotrypsin, alpha2-antiplasmin, alpha2-macroglobulin, inter-alpha-trypsin inhibitor and beta1-anticollagenase (collagenase), and, (iv) if the albumin-containing fraction used at step (i) is not a Fraction V precipitate of a Cohn/Oncley industrial plasma fractionation or an equivalent fraction of a different plasma fractionation process, fractionating the fraction to obtain a Fraction V precipitate or an equivalent fraction of a different plasma fractionation process;
the first albumin-containing fraction of human plasma is not subjected to said extracting; and
pasteurizing the albumin preparation.

29. (canceled)

30. The method according to claim 1, wherein the albumin preparation has an albumin content of at least 5% w/v, a C1E-inhibitor activity of greater than 0.0025 IU per kg total protein, and a PKA activity of less than or equal to 35 IU/ml.

31. The method according to claim 1, wherein the method comprises mixing a first albumin-containing fraction of human plasma with a second albumin-containing fraction of human plasma to obtain the albumin preparation, wherein the first albumin-containing fraction of human plasma is obtained by a process comprising extracting a first amount of C1E-inhibitor from an albumin-containing fraction of human plasma, and wherein the second albumin-containing fraction of human plasma is obtained by a process comprising extracting a second amount of C1E-inhibitor from an albumin-containing fraction of human plasma.

32-33. (canceled)

34. A human albumin preparation having an albumin content of at least 5% w/v, a C1E-inhibitor activity of greater than 0.0025 IU per kg total protein, and a PKA activity of less than or equal to 35 IU/ml.

35-44. (canceled)

45. A human albumin preparation prepared by the method according to claim 31.
